(19) United States
(12) Patent Application Publication
(10) Pub. No.: US 2024/0042103 A1
(43) Pub. Date: Feb. 8, 2024

(54) HEPARINIZED SMALL-DIAMETER VASCULAR GRAFTS

(71) Applicant: The Johns Hopkins University, Baltimore, MD (US)

(72) Inventors: Sharon Gerecht, Baltimore, MD (US); Morgan B. Elliott, Baltimore, MD (US); Hai-Quan Mao, Baltimore, MD (US); Theresa Chen, Baltimore, MD (US); Khyati Prasad, Baltimore, MD (US)

(21) Appl. No.: 18/256,677

(22) PCT Filed: Dec. 13, 2021
(86) PCT No.: PCT/US21/63128

§ 371 Date: Jun. 9, 2023

Related U.S. Application Data
(60) Provisional application No. 63/124,202, filed on Dec. 11, 2020.

(51) Int. Cl. A61L 27/54; A61L 27/50; A61L 27/22; A61L 33/00; B29D 23/00

(52) U.S. Cl. CPC A61L 27/54; A61L 27/507; A61L 27/225; A61L 33/0017; A61L 33/0076; B29D 23/00; B29K 2105/122

(57) ABSTRACT

Described are methods for embedding one or more therapeutic agents into vascular grafts and other scaffold-based devices, and methods of implanting vascular grafts comprising tubular scaffolds into subjects. The tubular scaffolds comprise hydrogel nanofibers that have internally aligned polymer chains and may contain one or more therapeutic agents.

### STATEMENT OF GOVERNMENTAL INTEREST

This invention was made with government support under grant nos. CBET1054415 and DMR1410240 awarded by the National Science Foundation. The government has certain rights in the invention.

### BACKGROUND

Coronary artery disease (CAD) is a leading cause of death or impaired quality of life for millions of individuals, resulting in more than half a million coronary artery bypass surgeries per year, Gui et al., 2009; Sundaram et al., 2014; Thompson et al., 2002; Mozaffarian et al., 2015, with treatment costs of over $100,000 per procedure. Gokhale, 2013. The standard treatment for CAD, which afflicts small-diameter arteries, is the use of autologous tissue as a bypass graft. Gui et al., 2009.

Autografts, however, have several disadvantages, including the requirement of a secondary surgical site to harvest the donor graft, as well as insufficient availability in patients with widespread atherosclerotic vascular disease or previously harvested vessels. While artificial grafts made of Gore-Tex®, Dacron®, and polyurethanes are the most common for vascular bypass surgeries that require grafts greater than 6 mm in diameter, synthetic polymer small diameter arterial grafts (sdVG, less than 6 mm in diameter) have yet to show clinical effectiveness. Lee et al., 2014. Despite the need for, and extensive literature on, sdVGs, Buttafoco et al., 2006; Zhang et al., 2009; Williams and Wick, 2004; Neumann et al., 2003; Hahn et al., 2007, a functional graft has remained elusive due to post-implantation challenges, including thrombogenicity, poor mechanical properties, aneurysmal failure, calcification, and intimal hyperplasia. Buttafoco et a 1., 2006; Hahn et al., 2007; Niklason et al, 1999.

Graft failure due to thrombosis is a key impediment and common challenge for clinical translation of engineered grafts, likely due to the lack of endothelial barrier function. Bilodeau et al., 2005; Sivarapatna et al., 2015. Systemic combination antithrombotic drug therapy treatments are not useful in clinical applications due to increased bleeding complications. Hess et al., 2017. Meanwhile, it has previously been shown that heparin-coated vascular stents minimally improve outcomes for CAD patients and that these coatings can be unreliable. Haude et al., 2003. A more applicable, local delivery approach is needed to minimize thrombosis in vascular grafts.

### SUMMARY

In some aspects, the presently disclosed subject matter provides a method for preparing a vascular graft, the method comprising: (a) conjugating one or more therapeutic agents to a protein to form a therapeutic agent-protein conjugate; (b) electrospinning a mixture of the therapeutic agent-protein conjugate and the protein to form a plurality of microfibers having the one or more therapeutic agents embedded therein; (c) forming one or more sheets of the plurality of microfibers having the one or more therapeutic agents embedded therein; and (d) forming a hollow tube comprising the one or more sheets of the plurality of microfibers having the one or more therapeutic agents embedded therein.

In some aspects, the one or more therapeutic agents comprises a compound having at least one carboxyl group. In some aspects, the one or more therapeutic agents is selected from the group consisting of an anticoagulant, an antiplatelet, an antihistamine, an antihypertensive, a nonsteroidal anti-inflammatory drug (NSAID), a statin, an antibiotic, a growth factor, factor Xa inhibitors, direct thrombin inhibitors, an anti-proliferative drug, and combinations thereof. In certain aspects, the anticoagulant comprises heparin. In particular aspects, the heparin comprises a low molecular weight heparin (LMWH). In more particular aspects, the LMWH is selected from the group consisting of bemiparin, nadroparin, reviparin, enoxaparin, parnaparin, certoparin, dalteparin, tinzaparin, ardeparin, and pharmaceutically acceptable salts and combinations thereof.

In some aspects, the protein is selected from the group consisting of fibrinogen, collagen, elastin, gelatin, hyaluronic acid, and combinations thereof.

In some aspects, the mixture of the therapeutic agent-protein conjugate is electrospun into a rotating bath. In some aspects, the one or more therapeutic agents comprises a LMWH, the protein comprises fibrinogen, and the rotating bath comprises thrombin, thereby forming a heparinized fibrin microfiber. In certain aspects, the method further comprises rastering a spinneret back and forth, for example along a linear platform, to form the sheet of microfibers having the one or more therapeutic agents embedded therein.

In some aspects, the method further comprises rolling the one or more sheets of microfibers having the one or more therapeutic agents embedded therein to form the hollow tube. In certain aspects, the method further comprises combining or alternating one or more sheets of microfibers having the one or more therapeutic agents embedded therein with one or more sheets comprising the protein alone, or sheets comprising one or more additional therapeutic agents.

In some aspects, the one or more therapeutic agents comprises a low molecular weight heparin (LMWH) and the protein comprises fibrinogen, and the method further comprises activating the LMWH and then conjugating the activated LMWH with the fibrinogen to form a LMWH-fibrinogen conjugate. In certain aspects, the LMWH is activated with 1-ethyl-3-[3-dimethylaminopropyl]carbodiimide hydrochloride (EDC)/N-hydroxysuccinimide (NHS).

In some aspects, the LMWH-fibrinogen conjugate is purified by centrifugal filtration and dialysis to remove non-conjugated LMWH. In certain aspects, the dialysis comprises a first solution comprising saline and a second solution against which the dialysis occurs comprising reverse osmosis (RO) H₂O. In more certain aspects, the dialysis comprises a first solution comprising sucrose, polyethylene oxide (PEO), or a combination of sucrose and PEO in saline and a second solution against which the dialysis occurs comprising sucrose, PEO, or a combination of sucrose and PEO in RO H₂O.

In some aspects, the method further comprises freezing and lyophilizing the purified LMWH-fibrinogen conjugate to form a powdered LMWH-fibrinogen conjugate.

In other aspects, the presently disclosed subject matter provides a vascular graft, microfibers, sheet, hollow tube, or mesh prepared by any of the presently disclosed methods.

In some aspects, the presently disclosed subject matter provides a vascular graft comprising one or more sheets or hollow tubes comprising a plurality of microfibers having one or more therapeutic agents embedded therein.

In some aspects, the one or more therapeutic agents comprises a compound having at least one carboxyl group. In certain aspects, the one or more therapeutic agents is selected from the group consisting of an anticoagulant, an antiplatelet, an antihistamine, an antihypertensive, a nonsteroidal anti-inflammatory drug (NSAID), a statin, an antibiotic, a growth factor, factor Xa inhibitors, direct thrombin inhibitors, an anti-proliferative drug, and combinations thereof.

In particular aspects, the anticoagulant comprises heparin. In certain aspects, the heparin comprises a low molecular weight heparin (LMWH). In more certain aspects, the LMWH is selected from the group consisting of bemiparin, nadroparin, reviparin, enoxaparin, parnaparin, certoparin, dalteparin, tinzaparin, ardeparin, and pharmaceutically acceptable salts and combinations thereof.

In some aspects, the plurality of microfibers further comprise a protein selected from the group consisting of fibrinogen, collagen, elastin, gelatin, hyaluronic acid, and combinations thereof.

In some aspects, the vascular graft comprises a tubular scaffold comprising a hollow core surrounded by one or more sheets comprising a plurality of microfibers having one or more therapeutic agents embedded therein. In certain aspects, the hollow core has an inner diameter having a range from about 0.1 mm to about 6 mm. In certain aspects, the one or more sheets have a combined thickness having a range from about 5 nm to about 10,000 μm.

In yet other aspects, the presently disclosed subject matter provides a method for treating vascular damage, the method comprising administering a vascular graft disclosed herein or prepared by any of the methods disclosed herein, to a subject having vascular damage.

In some aspects, the vascular graft is administered by vascular bypass surgery. In some aspects, the vascular damage is to an artery or vein. In some aspects, the vascular damage is caused by a disease or trauma. In certain aspects, the disease is selected from the group consisting of congenital cardiovascular defect (CCD), coronary artery disease (CAD), or peripheral artery disease (PAD).

In some aspects, the presently disclosed subject matter provides a kit comprising a powdered LMWH-fibrinogen conjugate, or reagents for preparing the powdered LMWH-fibrinogen conjugate, and solvents for reconstituting the powdered LMWH-fibrinogen conjugate for use in electrospinning.

In some aspects, the presently disclosed subject matter provides a kit comprising a vascular graft or scaffold prepared by the presently disclosed methods, wherein the vascular graft or scaffold is in a dehydrated or hydrated state, and optionally solutions for rehydrating the vascular grafts or scaffolds before use.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 illustrates a schematic of one embodiment of the presently disclosed process to fabricate heparinized sdVGs. First, low molecular weight heparin (LMWH) is conjugated to fibrinogen. Then, a mixture of LMWH-fibrinogen and fibrinogen is electrospun into a rotating thrombin bath to generate anticoagulant embedded fibrin microfibers. The electrospinning needle is rastered back and forth to fabricate a sheet of heparinized fibrin microfibers. The microfiber sheets are finally rolled around a mandrel to create hollow, hydrogel microfiber tubes, or the heparinized sdVG (from Elliott et al., 2019);

FIG. 2 illustrates a schematic of one embodiment of the presently disclosed process to conjugate LMWH and fibrinogen. First, LMWH is activated with EDC/NHS in an MES buffer solution overnight. Then, the fibrinogen is conjugated to the LMWH by carbodiimide chemistry in a 6.7× saline solution for 48 hours. The LMWH-fibrinogen is purified by centrifugal filtration and dialysis to remove non-conjugated LMWH and saline, respectively. Lastly, the LMWH-fibrinogen solution is frozen and lyophilized to yield a powder that can subsequently be used in the electrospinning process for the generation of heparinized sdVGs;

FIG. 3A and FIG. 3B illustrate the fabrication and some potential combinations of fibrin and heparinized fibrin sheets. (FIG. 3A) Pure fibrin (left) or heparinized fibrin (right) electrospun sheets were generated (black arrows indicate inner border). These sheets were wrapped onto two-dimensional (2D) frames (insets). (FIG. 3B) Fibrin sheets can be used to fabricate fibrin only sdVGs, as previously described (left). Heparinized fibrin sheets can be used to generate full thickness drug loaded sdVGs (right). The fibrin and heparinized fibrin sheets also can be combined or alternated, which enables the precise control of drug location and concentration within the hydrogel scaffold;

FIG. 4A, FIG. 4B, FIG. 4C, and FIG. 4D illustrate successful conjugation of LMWH-fibrinogen. Proton NMR (HNMR) of (FIG. 4A) control fibrinogen, (FIG. 4B) control LMWH, and (FIG. 4C) LMWH-fibrinogen (LMWH-F) samples. The unique peaks to LMWH are 4.40-3.5 ppm and 3.25-3.10 ppm, which are indicated by yellow boxes. (FIG. 4D) The absolute integrals of the HNMR curve were quantified and indicate successful conjugation of LMWH to fibrinogen. Yellow stars indicate significance relative to fibrinogen controls (set at 1), while black stars indicate significance between groups. **p<0.01 and ****p<0.0001 for 2-way ANOVA (n=3-6);

FIG. 5A, FIG. 5B, and FIG. 5C illustrate successful glycosylation of fibrinogen with LMWH. Sodium dodecyl sulfate polyacrylamide gel electrophoresis (SDS PAGE) was performed on LMWH-fibrinogen (LMWH-F) and fibrinogen. Fibrinogen conjugated to nadorparin calcium (N) or enoxaparin sodium (E) were both assessed. (FIG. 5A) Glycoprotein sugars were stained pink, then (FIG. 5B) proteins were stained blue. The alpha, beta, and gamma chains of fibrinogen are 63.5 kDa, 56 kDa, and 47 kDa, respectively. The fibrinogen soluble dimer is 340 kDa. The beta and gamma chains are indicated by orange boxes. (FIG. 5C) The intensity of the staining was quantified and indicates the gamma chain of fibrinogen had increased glycosylation after LMWH conjugation. Yellow stars indicate significance relative to fibrinogen controls (set at 1). * p<0.05 for 2-way ANOVA (n=3);

FIG. 6A, FIG. 6B, and FIG. 6C illustrate the reduced thrombogenicity of flat heparinized scaffolds relative to fibrin scaffolds. (FIG. 6A) Fibrin or heparinized fibrin sheets were wrapped onto 2D frames and incubated in porcine platelet rich plasma (pPRP). (FIG. 6B) Three-dimensional (3D) reconstruction images of platelets, which are anuclear and filamentous actin (F-actin) positive, attached to scaffolds. F-actin in green and nuclei in blue. (FIG. 6C) Reduced porcine platelet adhesion on heparinized scaffolds was demonstrated. N.S. is no significance in t-test (n=3);

FIG. 7A and FIG. 7B illustrate reduced thrombogenicity of heparinized sdVGs. Maximum intensity projection images of adhered human platelets on (FIG. 7A) fibrin and (FIG. 7B) heparinized fibrin sdVGs with 0.6-mm inner diameter. F-actin in green and CD41 (pre-activation platelet surface marker) in magenta. Scale bars are 200 μm. Lumen (L) and outer edges of the graft (white lines) are indicated;

FIG. 8A, FIG. 8B, FIG. 8C, and FIG. 8D illustrate reduced thrombogenicity of both partial and full thickness heparinized sdVGs. 3D reconstruction images of adhered human platelets on the luminal surface of (FIG. 8A) fibrin, (FIG. 8B) partial thickness heparinized, and (FIG. 8C) full thickness heparinized sdVGs with 5-mm inner diameter. For the partial thickness heparinized graft, only the innermost 6 sheets wrapped around the mandrel were heparinized, which was approximately 15% of the scaffold. F-actin in green. Lumen (L) is indicated. The scaffold faintly auto-fluoresced blue. (FIG. 8D) Reduced platelet adhesion on all heparinized scaffolds was demonstrated (n=1-2);

FIG. 9A and FIG. 9B illustrate the schematic and outcomes for the interpositional porcine carotid artery study. (FIG. 9A) Heparinized and fibrin grafts with 5-mm inner diameter were implanted in the carotid arteries of pigs for 4 weeks and assessed for patency. (FIG. 9B) The ePTFE clinical control graft was occluded by post-operative (post-op) week 2. The heparinized grafts had slightly improved patency relative to fibrin grafts at 2 and 4 wks post-op;

FIG. 10A, FIG. 10B, FIG. 10C, and FIG. 10D illustrate the patency of fibrin grafts implanted in the interpositional porcine carotid artery model. (FIG. 10A) Fibrin graft prior to harvest (top) and immediately after blood flow was re-established during surgery (bottom). sdVG patency was assessed at (FIG. 10B) 2 and 4 wks by color flow Doppler and (FIG. 10C) 4 wks by magnetic resonance imaging (MRI). Patent (P) and occluded (O) sdVGs are indicated by yellow arrows on the MRI. (FIG. 10D) Thrombus formation was grossly visible in the harvested, occluded fibrin graft at 4 weeks (1 of 2);

FIG. 11A, FIG. 11B, FIG. 11C, and FIG. 11D illustrate the patency of heparinized grafts implanted in the interpositional porcine carotid artery model. (FIG. 11A) Heparinized graft prior to harvest (top) and immediately after blood flow was re-established during surgery (bottom). sdVG patency was assessed at (FIG. 111B) 2 and 4 wks by color flow Doppler and (FIG. 11C) 4 wks by MRI. Patent (P) and occluded (O) sdVGs are indicated by yellow arrows on the MRI. (FIG. 11D) The open lumen was grossly visible in the harvested, patent heparinized grafts at 4 weeks (3 of 4);

FIG. 12A, FIG. 12B, and FIG. 12C illustrate a schematic of alterations to the conjugation of LMWH and fibrinogen to improve solubility. (FIG. 12A) The solution in the dialysis tubing was altered to be 100-mM sucrose in 0.2% polyethylene oxide (PEO) in saline, instead of just saline. The solution against which the dialysis occurs also was altered to be 100-mM sucrose in 0.2% PEO in RO H₂O, instead of just RO H₂O. (FIG. 12B) The LMWH-fibrinogen dialyzed against RO H₂O did not completely dissolve in 0.2% PEO in deionized (DI) H₂O (left). The LMWH-fibrinogen dialyzed against PEO and sucrose dissolved completely in DI H₂O to yield a final concentration of 3.64 mg/mL LMWH-F in 0.2% PEO (right). (FIG. 12C) The more soluble heparinized-fibrinogen mixed with fibrinogen was easily electrospun into heparinized fibrin sheets that were wrapped onto 2D frames, as previously described;

FIG. 13A, FIG. 13B, and FIG. 13C illustrate the further reduced thrombogenicity of heparinized scaffolds made from LMWH-fibrinogen with improved solubility. 2D sheets of fibrin, heparinized fibrin made from LMWH-fibrinogen dialyzed against PEO and sucrose (HF), or heparinized fibrin made from LMWH-fibrinogen with reduced solubility dialyzed against RO H₂O only (HF RS) were incubated in pPRP. The pPRP supernatant was subsequently analyzed for (FIG. 13A) peak thrombin generation, (FIG. 13B) time to peak thrombin generation, and (FIG. 13C) the steepest rate of thrombin generation. Collagen I (Col I) coated glass coverslips were used as a positive control. The HF scaffolds appear to reduce the rate of thrombin generation relative to fibrin and HF RS scaffolds. *p<0.05, **p<0.01, and ****p<0.0001 for 1-way ANOVA (n=2-5);

FIG. 14A, FIG. 14B, FIG. 14C, and FIG. 14D show the effects of long-term storage on fibrin hydrogel microfiber tubes (FMTs) not biologically significant. FMTs were stored in a dehydrated state for 1, 3, 6, or 12 months in the (FIG. 14A) freezer, refrigerator, or room temperature. After storage, the FMTs were rehydrated and (FIG. 14B) underwent circumferential tensile testing. (FIG. 14C) FMT (i) stiffness, (ii) swelling ratio, and (ii) wall thickness relative to control FMTs, which were tested within 5 days of dehydration. (FIG. 14D) Mechanical properties, including (i) circumferential UTS, (ii) circumferential STF, and (iii) modulus of toughness, of stored FMTs compared to control FMTs and the native mouse abdominal aorta (AAo). Black and yellow stars indicate significance over time and between groups, respectively. n=4-13, *p<0.05, **p<0.01, ***p<0.001, ****p<0.0001;

FIG. 15A, FIG. 15B, and FIG. 15C demonstrate that fibrin hydrogel microfiber tube mechanical properties unaffected by rehydration time and accurately predicted by accelerated aging model. (FIG. 15A) (i) Circumferential UTS, (ii) modulus of toughness, (iii) circumferential STF, and (iv) stiffness of FMTs that underwent circumferential tensile testing 1, 4, and 7 hours after rehydration (n=6). (FIG. 15B) Using the (i) ASTM International accelerating aging model and a conservative aging factor, we calculated the (ii) time needed to store FMTs at elevated temperatures to simulate longer-term storage at reduced, ambient temperatures. (FIG. 15C) Model reliability is indicated by comparing the mechanical properties, including (i) circumferential UTS, (ii) modulus of toughness, (iii) circumferential STF, and (iv) stiffness of FMTs at elevated (e) and ambient (a) temperatures. Black stars indicate significance between elevated and ambient temperature groups (n=7-16). N.S. is no significance, **p<0.01, ***p<0.001, ****p<0.0001;

FIG. 16A, FIG. 16B, FIG. 16C, FIG. 16D, FIG. 16E, FIG. 16F, and FIG. 16G illustrate the heparinized fibrin microfiber tube fabrication, drug release, and mechanical properties. (FIG. 16A) Schematic of fabricating heparinized fibrin (HF) tubes, which involves conjugating LMWH to fibrinogen, electrospinning a mixture of LMWH-fibrinogen and fibrinogen into anticoagulant embedded microfibers, and rolling microfiber sheets around a mandrel to create hollow, hydrogel microfiber tubes. HF and Fibrin tubes were assessed with (FIG. 16B) DMMB for sulfated glycosaminoglycan (GAG) content (n=6-9), modified DMMB and TP assays for (FIG. 16C) drug release in PBS (n=6-18) and (FIG. 16D) enzymatic drug release (n=6-12). Black and colored stars indicate significance between groups and over time, respectively. Colored arrows indicate time of complete degradation. The scaffolds were also assessed for (FIG. 16E) swelling ratio (n=18-43), (FIG. 16F) wall thickness, and (FIG. 16G) mechanical properties (n=3-4). N.S. is no significance, *p<0.05, **p<0.01;

FIG. 17 shows the synthesis of LMWH-Fibrinogen;

FIG. 18A, FIG. 18B, and FIG. 18C demonstrate the reduced thrombogenicity of heparinized fibrin scaffolds. (FIG. 18A) Porcine and human PRP were incubated on 2D heparinized fibrin (HF) scaffolds, Fibrin scaffolds, and collagen I (Col I, positive control) to assess (FIG. 18B) platelet adhesion and (FIG. 18C) thrombin generation. n=6-16, N.S. is no significance, *p<0.05, **p<0.01, ***p<0.001, ****p<0.0001;

FIG. 19A, FIG. 19B, FIG. 19C, and FIG. 19D demonstrate the fabrication of sdVGs with a size suitable for human application. (FIG. 19A) (i) Schematic comparing the size of FMTs, which were increased from (ii) 0.6 mm to (iii) 5 mm inner diameters by simply changing the mandrel size used to collect fibrin microfiber sheets. Representative SEM micrographs of the external surface of FMTs with controlled, longitudinally aligned fibrin microfibers at (iv) low (scale bar: 200 μm) and (v) high (scale bar: 20 μm) magnification. (FIG. 19B) Optimization of PCL surgical sheath suture retention strength (SRS) to match the native porcine carotid artery by adjusting the air gap distance used during electrospinning, measured before and after heat treatment (HT) (n=5-36). (FIG. 19C) Fibrin-PCL sdVG prepared for large animal implantation. (FIG. 19D) Mechanical properties of Fibrin-PCL sdVG, HF-PCL sdVG, PCL surgical sheath, native porcine vessel controls, and GORE® PROPATEN® clinical control (n=3-6). Graft configuration diagrams indicate fibrin (grey), LMWH (black), and PCL sheath (green) (not to scale). *p<0.05, **p<0.01, ***p<0.001, ****p<0.0001;

FIG. 20A, FIG. 20B, and FIG. 20C show the fabrication and optimization of ultra-thin PCL surgical sheath. (FIG. 20A) Electrospinning of PCL with adjustable air gap distance (AGD). (FIG. 20B) Humidity during electrospinning does not affect PCL suture retention strength (SRS) (n=2-12). (FIG. 21C) PCL heat treatment set-up;

FIG. 21A, FIG. 21B, FIG. 21C, FIG. 21D, and FIG. 21E illustrate the extended patency of heparinized grafts and remodeling of Fibrin- and HF-PCL sdVGs in vivo. (FIG. 21A) Grafts were (i) implanted in a carotid artery (CA) interposition porcine model and (ii) maintained hemostasis without rupture. (FIG. 21B) (i) Summary of sdVG and clinical control graft patency at 2 weeks post-implantation, measured by sonography. (ii) Color Doppler visualizing blood flow. Lack of color indicates loss of patency, while blue or red indicates blood flow in the lumen (L). Graft walls (W) are indicated. (FIG. 21C) Representative cross-sectional histological sectioning of harvested, patent grafts at 4-5 weeks post-implantation and control native CA. Staining for H&E, Masson's Trichrome, SMCs (αSMA), von Kossa, and ECs (CD31). Boxes in the low magnification images indicate the high magnification image (inset) locations (n=1-3). Lumen (L), fibrin layer (F), PCL sheath (P), GORE® PROPATEN® scaffold (G), sutures (S), and individual CD31 positive cells (arrowheads) are indicated. (FIG. 21D) Dimensions and (FIG. 21E) mechanical properties of harvested HF- and Fibrin-PCL sdVG grafts compared to pre-implant sdVG, GORE® PROPATEN®, and native porcine CA controls. Rings from the anastomosed native CA were also assessed. Each dot represents an individual vessel, n=2-8, *p<0.05, **p<0.01, ***p<0.001, ****p<0.0001; and

FIG. 22 shows the histology of occluded grafts. Representative cross-sectional histological sectioning of harvested, occluded grafts at 4-5 weeks post-implantation. Staining for H&E, Masson's Trichrome, SMCs (αSMA), and von Kossa. Boxes in the low magnification images indicate the high magnification image (inset) locations (n=1-3). Lumen (L), fibrin layer (F), PCL sheath (P), and GORE-TEX® ePTFE scaffold (G) are indicated.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Heparinized Small-Diameter Vascular Grafts

To overcome limitations of vascular grafts known in the art, including thrombus formation, the presently disclosed subject matter provides a technique to conjugate drugs to the proteins used in the electrospinning process and fabricate grafts wherein the drugs, such as low molecular weight heparin (LMWH), are conjugated within the microfiber scaffold. LMWHs are anticoagulant drugs used in combination with dual-antiplatelet therapy (DAPT) clinically to treat acute coronary syndrome. Ostadal et al., 2008; Heart.org (2017). LMWHs are safer and more effective than unfractionated heparin. Ostadal et al., 2008; Tasatargil et al., 2005.

The presently disclosed method of sustained delivery of anti-coagulant drugs via controlled locations and dosages within the sdVG will provide a more effective and safer approach to alleviate acute clot formation. This approach will overcome the significant drawbacks of global heparin therapy and heparin coating of vascular grafts.

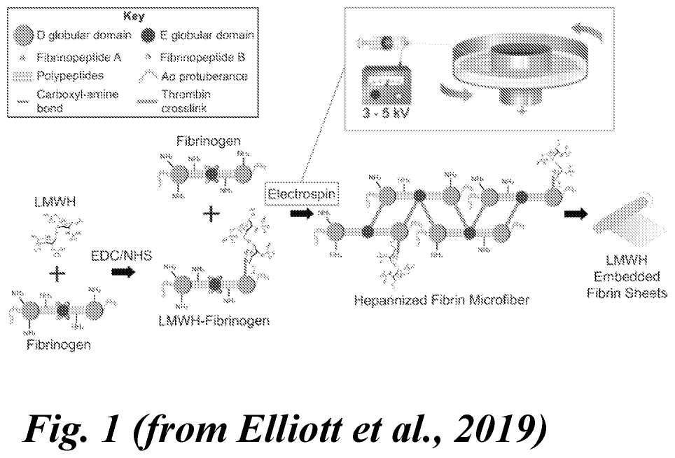

Referring now to FIG. 1, the presently disclosed approach embeds drugs in the microfiber scaffold using a unique electrospinning process, thereby creating grafts with low molecular weight heparin (LMWH) chemically conjugated to the scaffold (FIG. 1). Chemically conjugating the LMWH to the protein backbone of the natural polymer scaffold enables not only controlled dosage delivery, but also sustained release of the drug while the scaffold degrades, enabling the generation of heparinized sdVGs for populations with a high risk of thrombus formation.

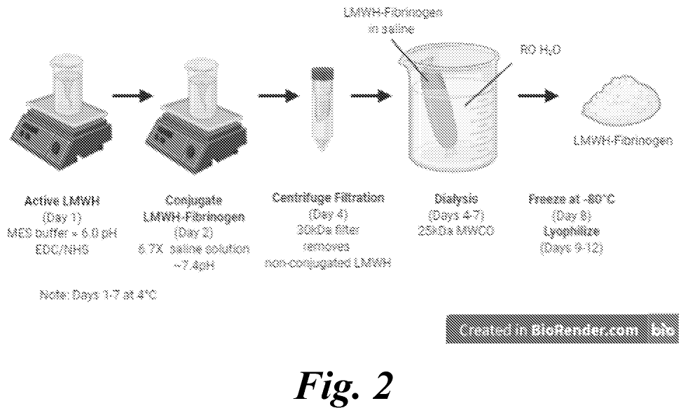

Fabrication of LMWH-embedded sdVGs first requires synthesis of LMWH-fibrinogen (LMWH-F), which involves conjugation of fibrinogen with LMWH using carbodiimide chemistry and purification of the LMWH-F to prevent bulk release of the anticoagulant into systemic circulation (FIG. 2). Yang et al., 2010.

The conjugation of LMWH to fibrinogen was enhanced by first using an elemental analysis to ensure the ratio of carboxyl groups to EDC/NHS was ideal, which resulted in increasing the concentrations of EDC and NHS for carbodiimide crosslinking. Additionally, the LMWH was set to be in large molar excess to fibrinogen (46×). Due to LMWH (mean molecular weight (MW) 4.5 kDa) being a highly negatively charged molecule, Ostadal et al., 2008; Zhang et al., 2010; Ouyang et al., 2019; Barradell and Buckley, 1992, centrifugal filtration through a 30 kDa filter was used to remove non-conjugated LMWH, while dialysis through 25 kDa MWCO tubing was primarily used to remove saline from the LMWH-F solution. Lyohpilization of the synthesized compound enabled storage for later use. The percent yield of LMWH-F using this synthesis protocol was 63.89±12.46% (n=11). The modified synthesis protocol significantly improved the LMWH concentration from the previously published 42.73 mg/g to 551.72±438.83 mg/g (n=7). Yang et al., 2010.

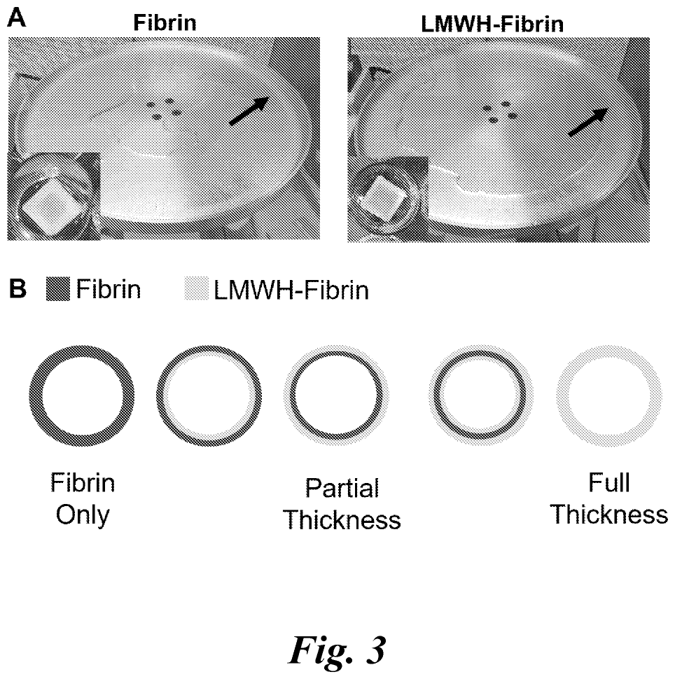

With the presently disclosed electrospinning process, the location of the drug within the graft can be controlled by modulating which of the longitudinally or circumferentially oriented electrospun fibrin sheets wrapped around the mandrel contain LMWH (FIG. 3). The concentration of LMWH in the sdVG can be controlled by not only altering the ratio of LMWH-F:fibrinogen used in electrospinning, but also by changing the number of fibrin sheets that contain LMWH-F.

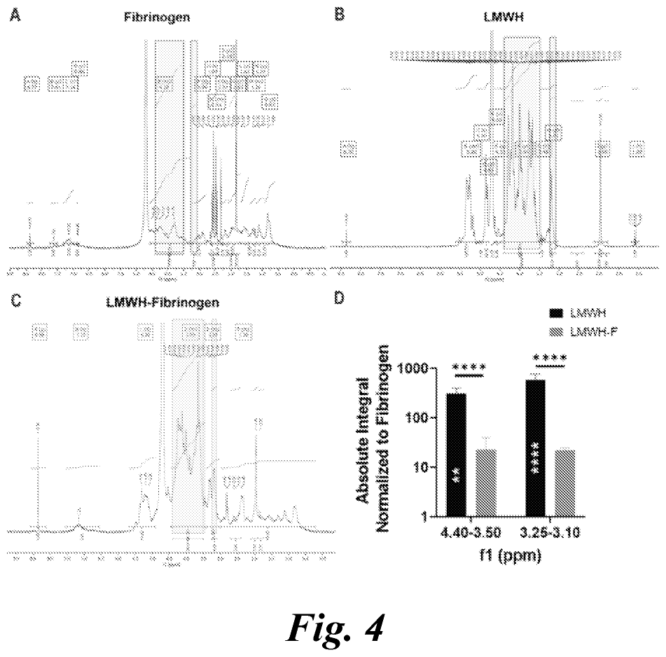

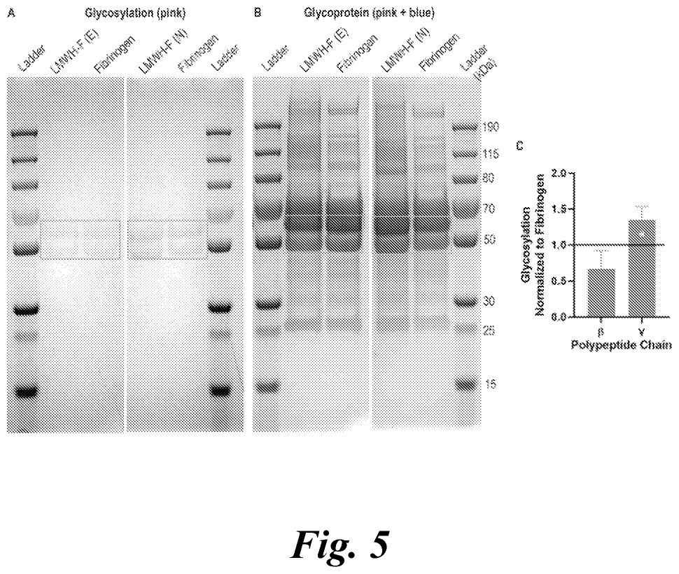

HNMR and SDS PAGE were used to assess the LMWH-F conjugation. Zhang et al., 2010. HNMR indicates that the LMWH has unique peaks at 4.40-3.50 and 3.25-3.10 ppm relative to fibrinogen. These peaks were 22 times higher in the LMWH-F compared to the fibrinogen control (FIG. 4). Glycoprotein staining of the SDS PAGE indicated that the γ-chain of fibrinogen has 1.35 times increased glycosylation after the synthesis (FIG. 5). Both of these tests indicate that LMWH, which is a glycosaminoglycan, was successfully bound to the fibrinogen protein. The glycoprotein staining also demonstrates that the synthesis can be performed with multiple LMWHs, including clinically used nadroparin calcium (N) and enoxaparin sodium (E). These LMWH-fibrinogen compounds have similar banding on the SDS PAGE, which slightly differs from the pure fibrinogen. These LMWHs both contain carboxyl groups and have similar pharmacodynamic characteristics. Ostadal et al., 2008; Ouyang et al., 2019; Barradell and Buckley, 1992. Therefore, drugs with a carboxyl group can be conjugated to the protein backbone of the scaffold by using carbodiimide crosslinking.

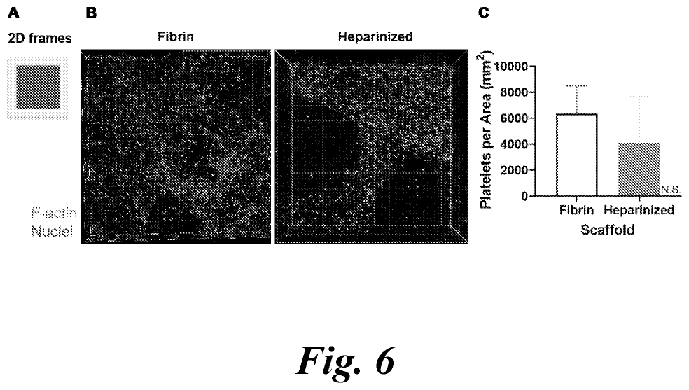

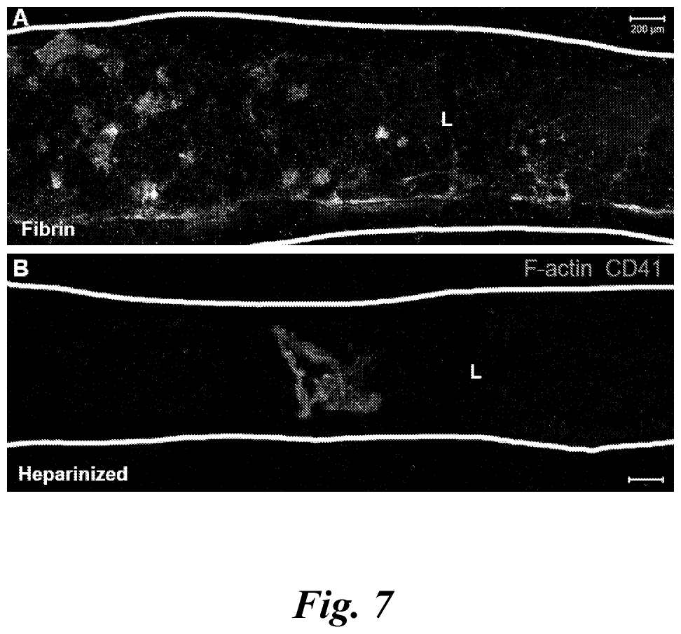

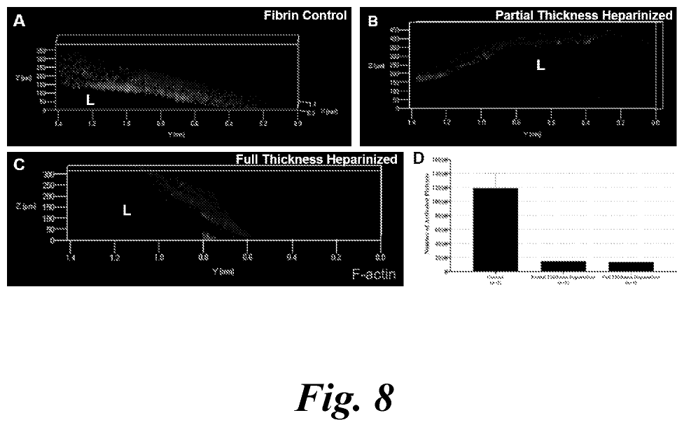

Dynamic incubation of porcine PRP with 0.5-U/mL thrombin for 1 hr at 37° C. on electrospun sheets made with pure fibrinogen or 40% LMWH-F was used as an in vitro thrombogenesis assay. Stevens, 2004; Badimon et al., 2012. The number of activated platelets adhered to the pure fibrin sheets was 1.5 times higher than the 40% heparin-fibrin sheets (FIG. 6), indicating the potential of our heparinized fibrin grafts to overcome the thrombogenicity challenge typically faced by synthetic sdVGs. Pashneh-Tala et al., 2015. Incubation of human PRP in the lumen of 0.6-mm and 5-mm inner diameter sdVGs indicated the heparinized sdVGs reduce platelet adhesion to the luminal surface relative to fibrin sdVGs (FIG. 7-FIG. 8), reenforcing the clinical relevance of this embedded drug approach. The 5-mm inner diameter sdVGs also demonstrated the ability to control the location of heparinized fibrin (FIG. 8). The heparinized fibrin scaffolds reduce thrombogenicity in a variety of configurations.

The commonly used porcine model is excellent to assess graft function and clinical-applicability due to the pig's similarity with the human cardiovascular anatomy, physiology, and thrombosis mechanisms. Pashneh-Tala et al., 2015; Stacy et al., 2014; Hoerstrup et al., 2006. The porcine model will enable a more strict assessment of plaque formation and thrombogenicity than previously used mouse models, which have different clotting mechanisms than humans. Pashneh-Tala et al., 2015.

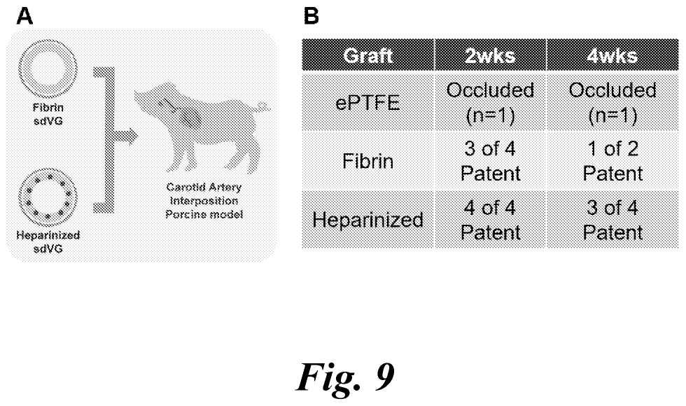

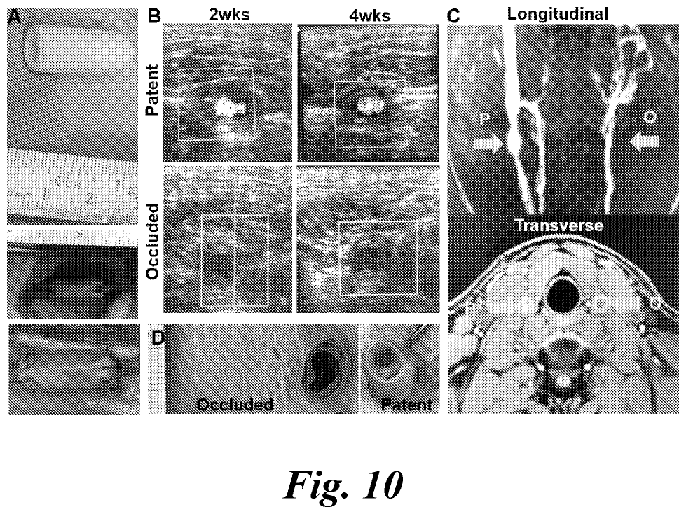

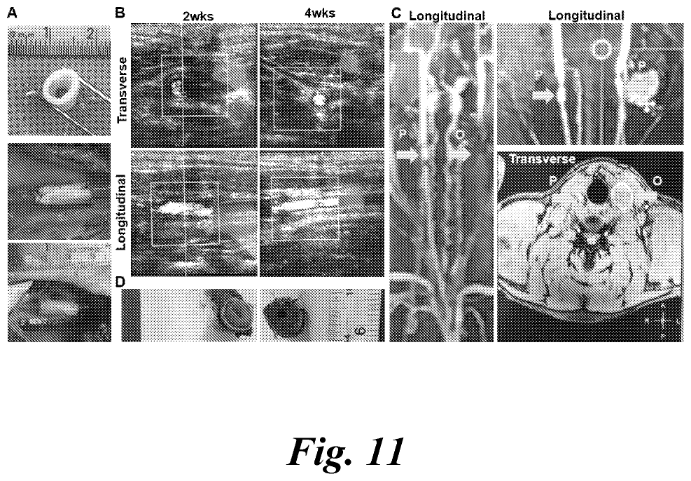

Heparinized and fibrin grafts were implanted in an interpositional porcine carotid artery model for 4 weeks (FIG. 9), as grafts undergo maximum thrombus formation during this period. Fleser et al., 2004. Using color flow Doppler, it was found that the clinical control ePTFE graft occluded within 2 weeks; meanwhile, the majority of fibrin and all heparinized sdVGs were patent at this time (FIG. 9, FIG. 10, and FIG. 11). Ultimately, the heparinized sdVGs had slightly improved patency relative to fibrin grafts at 2- and 4-weeks post-op. (FIG. 9, FIG. 10 and FIG. 11).

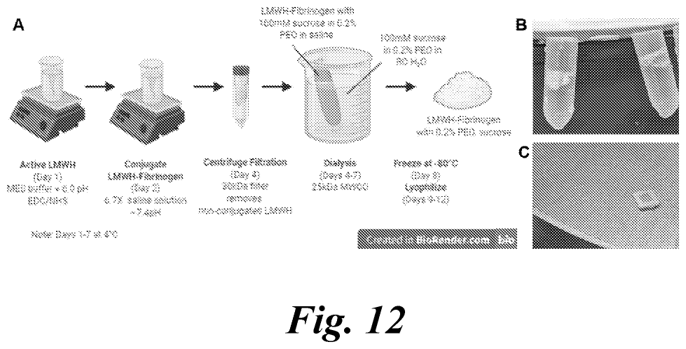

To further improve the patency of the heparinized sdVGs, the LMWH-fibrinogen synthesis process was further modified to improve the solubility of the glycoprotein (FIG. 12). In particular, the solution in the dialysis tubing was changed to be a final concentration of 100-mM sucrose in 0.2% PEO in saline. Previously, the dialysis of saline against RO H₂O caused the LMWH-F to precipitate during dialysis as saline was removed and the resultant glycoprotein was not completely soluble, which limited the amount of LMWH incorporated into the hydrogel scaffold. The sucrose was added to enhance the stability of the protein during the drying, storage, and moisture changes. Lee and Timasheff, 1981; Mensink et al, 2017.

Additionally, the PEO was added as this has been able to dissolve fibrinogen completely for electrospinning in the past, even in the absence of saline. Elliott et al., 2019. To ensure sucrose stayed in the dialysis tubing and to prevent excess osmosis, the solution against which the dialysis occurs was also altered to be 100-mM sucrose in 0.2% PEO in RO H₂O instead of just RO H₂O (FIG. 12).

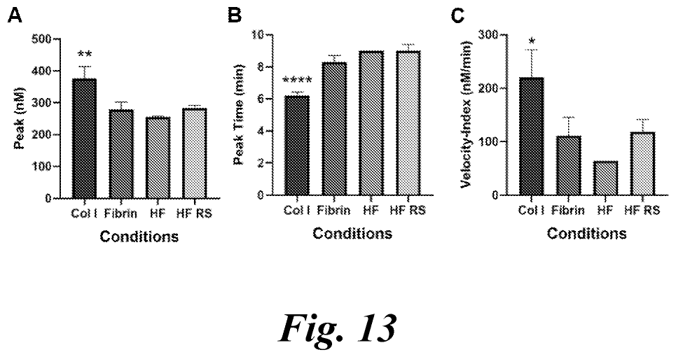

To assess if the improved solubility of LMWH-F reduced thrombogenicity, a thrombin generation assay was performed on porcine PRP with 0.1 U/mL thrombin that had been incubated on 2D sheets made of fibrin, heparinized fibrin made from LMWH-fibrinogen dialyzed against PEO and sucrose (HF), or heparinized fibrin made from LMWH-fibrinogen with reduced solubility dialyzed against RO H₂O only (HF RS). Collagen I coated glass coverslips (Col I) were used as a positive control. The more thrombogenic Col I samples had significantly increased peak thrombin generation, reduced time to peak thrombin generation, and faster rate of thrombin generation relative to all the samples. The HF scaffolds appear to reduce the rate of thrombin generation relative to fibrin and HF RS scaffolds (FIG. 13). It should be noted that the HF scaffolds only contained 18% LMWH-F, compared to the HF RS scaffolds that had 40% LMWH-F co-dissolved with fibrinogen. Therefore, the HF scaffolds had slightly reduced thrombogenicity using 22% less LMWH-F. Increasing the concentration of the LMWH-F can be achieved by increasing the centrifugal filtration time of the LMWH-F and is expected to drastically decrease scaffold thrombogenicity.

Accordingly, in some embodiments, the presently disclosed subject matter provides a method for preparing a vascular graft, the method comprising:
(a) conjugating one or more therapeutic agents to a protein to form a therapeutic agent-protein conjugate;
(b) electrospinning a mixture of the therapeutic agent-protein conjugate and the protein to form a plurality of microfibers having the one or more therapeutic agents embedded therein;
(c) forming one or more sheets of the plurality of microfibers having the one or more therapeutic agents embedded therein; and
(d) forming a hollow tube comprising the one or more sheets of the plurality of microfibers having the one or more therapeutic agents embedded therein.

In some embodiments, the vascular graft comprises a small diameter vascular graft (sdVG). As used herein, the term “small diameter vascular graft (sdVG)” is small-diameter vascular graft having an inner diameter less than about 6 mm. The vascular graft may taper or vary in size, including variations in length, diameter, and wall thickness, to match the existing vasculature and subject needs.

By “microfiber” is meant a solid tubular structure made up of a bundle of nanofibers.

A “tubular scaffold” generally means a structure comprising a sheet of nanofibers or microfibers forming a circumference around a hollow core.

In some embodiments, the one or more therapeutic agents comprises a compound having at least one carboxyl group. As used herein, the term “carboxyl group” is a functional group consisting of a carbon atom double-bonded to an oxygen atom and singly bonded to a hydroxyl group and comprises the R—C(═O)—OH group. Representative therapeutic agents having a carboxyl group include, but are not limited to, LMWH heparins, such a nadroparin calcium and enoxaparin sodium as disclosed herein; factor Xa inhibitors, such as fondaparinux, rivaroxaban, rapixaban and edoxaban; direct thrombin inhibitors, such as argatroban, inogatran, melagatran (and its prodrug ximelagatran), and dabigatran; antiplatelet drugs, such as clopidogrel and prasugrel, and antihypertension drugs, such as azilsartan, candesartan, eprosartan, irbesartan, losartan, olmesartan, telmisartan, and valsartan. The role of the carboxyl group in pharmaceutical compounds and representative pharmaceutical compounds having a carboxyl group are disclosed in Lamberth and Dinges, 2016, which is incorporated by reference in its entirety.

In some embodiments, the one or more therapeutic agents is selected from the group consisting of an anticoagulant, an antiplatelet, an antihistamine, an antihypertensive, a nonsteroidal anti-inflammatory drug (NSAID), a statin, an antibiotic, a growth factor, factor Xa inhibitors, direct thrombin inhibitors, an anti-proliferative drug like rapamycin, and combinations thereof. In certain embodiments, the anticoagulant comprises heparin. In particular embodiments, the heparin comprises a low molecular weight heparin (LMWH).

Heparin is a naturally occurring polysaccharide that inhibits coagulation. Natural heparin consists of molecular chains of varying molecular weights from about 5 kDa to over 40 kDa. In contrast, LMWHs consist of only short chains of polysaccharide and are defined as heparin salts having an average molecular weight of less than 8 kDa and for which at least 60% of all chains have a molecular weight less than 8 kDa. Representative embodiments of LMWH along with their average molecular weights are provided in Table 1.

TABLE 1
Representative LMWHs

| | Average molecular |
|---|---|
| LMWH | weight (Da) |
| Bemiparin | 3600 |
| Nadroparin | 4300 |
| Reviparin | 4400 |
| Enoxaparin | 4500 |
| Parnaparin | 5000 |
| Certoparin | 5400 |
| Dalteparin | 5000 |
| Tinzaparin | 6500 |
| Ardeparin | 5500-6500 |

Accordingly, in yet more particular embodiments, the LMWH is selected from the group consisting of bemiparin, nadroparin, reviparin, enoxaparin, parnaparin, certoparin, dalteparin, tinzaparin, ardeparin, and pharmaceutically acceptable salts and combinations thereof, including, for example sodium, potassium, calcium, ammonium, lithium, tosylates, and the like.

In some embodiments, the protein is selected from the group consisting of fibrinogen, collagen, elastin, gelatin, hyaluronic acid, and combinations thereof.

In some embodiments, the mixture of the therapeutic agent-protein conjugate is electrospun into a rotating bath.

In some embodiments, the one or more therapeutic agents comprises a LMWH, the protein comprises fibrinogen, and the rotating bath comprises thrombin, thereby forming a heparinized fibrin microfiber. In certain embodiments, the method further comprises rastering a spinneret, e.g., an electrospinning needle and the like, back and forth, for example along a linear platform, to form the sheet of microfibers having the one or more therapeutic agents embedded therein.

In some embodiments, the method further comprises rolling the one or more sheets of microfibers having the one or more therapeutic agents embedded therein to form the hollow tube. In certain embodiments, the method further comprises combining or alternating one or more sheets of microfibers having the one or more therapeutic agents embedded therein with one or more sheets comprising the protein alone, or sheets comprising one or more additional therapeutic agents.

In some embodiments, the one or more therapeutic agents comprises a low molecular weight heparin (LMWH) and the protein comprises fibrinogen, and the method further comprises activating the LMWH and then conjugating the activated LMWH with the fibrinogen to form a LMWH-fibrinogen conjugate. In certain embodiments, the LMWH is activated with 1-ethyl-3-[3-dimethylaminopropyl]carbodiimide hydrochloride (EDC)/N-hydroxysuccinimide (NHS).

In some embodiments, the LMWH-fibrinogen conjugate is purified by centrifugal filtration and dialysis to remove non-conjugated LMWH. In certain embodiments, the dialysis comprises a first solution comprising sucrose, polyethylene oxide (PEO), or a combination of sucrose and PEO in saline and a second solution against which the dialysis occurs comprising sucrose, PEO, or a combination of sucrose and PEO in RO H₂O.

In some embodiments, the method further comprises freezing and lyophilizing the purified LMWH-fibrinogen conjugate to form a powdered LMWH-fibrinogen conjugate.

In other embodiments, the presently disclosed subject matter provides a vascular graft, microfibers, including a solid bundle, sheet, hollow tube, or mesh prepared by any of the presently disclosed methods.

In some embodiments, the presently disclosed subject matter provides a vascular graft comprising one or more sheets or hollow tubes comprising a plurality of microfibers having one or more therapeutic agents embedded therein.

In some embodiments, the one or more therapeutic agents comprises a compound having at least one carboxyl group. In certain embodiments, the one or more therapeutic agents is selected from the group consisting of an anticoagulant, an antiplatelet, an antihistamine, an antihypertensive, a nonsteroidal anti-inflammatory drug (NSAID), a statin, an antibiotic, a growth factor, factor Xa inhibitors, direct thrombin inhibitors, an anti-proliferative drug, and combinations thereof.

In particular embodiments, the anticoagulant comprises heparin. In certain embodiments, the heparin comprises a low molecular weight heparin (LMWH). In more certain embodiments, the LMWH is selected from the group consisting of bemiparin, nadroparin, reviparin, enoxaparin, parnaparin, certoparin, dalteparin, tinzaparin, ardeparin, and pharmaceutically acceptable salts and combinations thereof.

In some embodiments, the plurality of microfibers further comprise a protein selected from the group consisting of fibrinogen, collagen, elastin, gelatin, hyaluronic acid, and combinations thereof.

In some embodiments, the vascular graft comprises a tubular scaffold comprising a hollow core surrounded by one or more sheets comprising a plurality of microfibers having one or more therapeutic agents embedded therein. In some embodiments, the hollow core has an inner diameter having a range from about 0.1 mm to about 6 mm, including 0.1, 0.2, 0.3, 0.4, 0.5, 0.6, 0.7, 0.8, 0.9, 1, 2, 3, 4, 5, and 6 mm.

In certain embodiments, the one or more sheets have a combined thickness having a range from about 5 nm to about 10,000 μm, including 5 nm, 10 nm, 50 nm, 100 nm, 500 nm, 1 μm, 10 μm, 100 μm, 500 μm, 1000 μm, 2000 μm, 3000 μm, 4000 μm, 5000 μm, 6000 μm, 7000 μm, 8000 μm, 9000 μm, and 10,000 μm.

In yet other embodiments, the presently disclosed subject matter provides a method for treating vascular damage, the method comprising administering a vascular graft disclosed herein or prepared by any of the methods disclosed herein, to a subject having vascular damage.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In some embodiments, the vascular graft is administered by vascular bypass surgery.

In some embodiments, the vascular damage is to an artery or vein.

In some embodiments, the vascular damage is caused by a disease or trauma. In certain embodiments, the disease is selected from the group consisting of congenital cardiovascular defect (CCD), coronary artery disease (CAD), or peripheral artery disease (PAD).

In some embodiments, the presently disclosed subject matter provides a kit comprising a powdered LMWH-fibrinogen conjugate, or reagents for preparing the powdered LMWH-fibrinogen conjugate, and solutions for reconstituting the powdered LMWH-fibrinogen conjugate for use in electrospinning. The kits also can include vascular grafts or scaffolds prepared by the presently disclosed methods, which can be either dehydrated or hydrated. In such embodiments, the kits also can include solutions for rehydrating the vascular grafts or scaffolds before use. The component(s) of the kits may be packaged either in aqueous media or in lyophilized form or frozen form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. Various combinations of components may be comprised in a single vial. The kits of the present invention also will typically include a means for containing the components of the kits and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The components of the kit may be provided as dried powder(s). When reagents and/or components are provided as a dry powder, the powder can be reconstituted by the addition of a suitable solvent. It is envisioned that the solvent may also be provided in another container means. The kit also can include instructions for use.

In sum, the presently disclosed methods can used to electrospin drug-conjugated proteins to make fibrin microfiber scaffolds, including individual microfibers, flat sheets, and hollow tubes. In general, any drug with a carboxyl group can be incorporated into the scaffold due to the use of carbodiimide chemistry. The graft prepared by the presently disclosed methods provide sustained, local drug (e.g., anticoagulant) release while the graft degrades. Varying concentrations of drug can be electrospun into the fibrin microfibers. The location of the drug and drug concentration within the scaffold can be controlled by modulating which sheets are used to build the scaffold. The embedded heparin remains functional after incorporation into the scaffold and will provide more reliable local administration of drugs, especially in a vascular setting.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods

### 1.1 Preparation of Fibrin Hydrogel Tubes

Fibrin hydrogel microfiber sheets were prepared as previously described by electrospinning 2.0 wt % fibrinogen solution co-dissolved in 0.2 wt % PEO in water under the effects of an applied electric field (4.5 kV) to propel the resultant fiber jet across an air gap of 2 cm and onto a rotating collection bath (45 rpm) containing 50-mM calcium chloride and 20-U/mL thrombin. Elliott et al., 2019. The landing position of the spinning jet was rastered back and forth via use of a linear stage during the spinning step to yield a uniform aligned fibrin sheet.

Hollow fibrin tubes with multidirectional alignment were formed by rolling sheets arranged first parallel, then perpendicular, and again parallel to the fiber orientation onto polytetrafluoroethylene (PTFE)-coated stainless-steel mandrels to generate tubes. This process created alternating layers of longitudinally, circumferentially, and longitudinally aligned fibrin microfibers. Tube wall thickness was controlled by altering the number of wraps around the mandrel. To alter the inner diameter of the graft, the diameter of the mandrel used to collect the fibrin sheets was changed. To increase the length of the graft, the width of the fibrin sheet was increased by increasing the path length of the rastering needle. Following wrapping, fibrin tubes were crosslinked for 15 hours in 40-mM EDC/100 mM NHS dissolved in PBS and dehydrated in a series of 25, 50, 60, 70, 80, 90, 95, 100, 100, and 100% EtOH solutions for a minimum of 15 minutes per step and then allowed to air dry. Dried fibrin tubes were removed from the PTFE mandrels following dehydration.

### 1.2 Synthesis of LMWH-Fibrinogen

LMWH was pre-activated in 0.05-M 2-morpholinoethanesulfonic acid (MES) in MilliQ H₂O (pH 6.0). The LMWH at 1 mg/mL was combined with 1.07-mM EDC and 1.17-mM NHS to activate overnight while stirring. Yang et al., 2010. To conjugate LMWH and fibrinogen (FIG. 2), the activated-LMWH solution was then diluted 1:3 in 10×PBS with dissolved fibrinogen for 2 days, resulting in a final concentration of 0.5 mg/mL fibrinogen and 6.7× saline (pH 7.4). Based on an elemental analysis, the concentrations of EDC and NHS were increased for carbodiimide crosslinking to ensure that LMWH was in large molar excess to fibrinogen (0.0741 mm vs. 0.0016 mM, respectively). Due to LMWH (mean MW 4.5 kDa) being a highly negatively charged molecule, Ostadal et al., 2008; Zhang et al., 2010; Ouyang et al., 2019; Barradell and Buckley, 1992, the purification was altered to include not only dialysis through 25 kDa MW cut off (MWCO) tubing against RO H₂O for 3 days, but also centrifugal filtration through a 30 kDa filter at 3500 g for 30 mins.

Alternatively, dialysis against 100-mM sucrose in 0.2% PEO in RO H₂O was performed to enhance the solubility of LMWH-Fibrinogen (LMWH-F) (FIG. 12A). For this dialysis, the LMWH-F solution was diluted 1:2 in 6.7×PBS with 200-mM sucrose and 0.4% PEO before being placed in the tubing. All synthesis steps were performed at 4° C. Lastly, the LMWH-F solution was frozen at −80° C. and lyophilized until dry for storage at 4° C. and future electrospinning. HNMR, glycoprotein staining (G-Biosciences) of SDS PAGE, and the colorimetric toluidine blue O (TBO) method were used to assess conjugation of LMWH to fibrinogen (FIG. 4 and FIG. 5). Zhang et al., 2010; Stevens, 2004; Jeon et al., 2006; Smith et al., 1980; Yao et al., 2005; Yang et al., 2010. Pure fibrinogen was used as a control.

### 1.3 Fabrication of Heparinized Fibrin Hydrogel Scaffolds

Fabrication of LMWH-embedded scaffolds involves synthesis of LMWH-F and co-dissolving LMWH-F with fibrinogen for electrospinning (FIG. 1). In representative embodiments presented herein, a mixture of 40% LMWH-F and 60% fibrinogen co-dissolved in 0.2% PEO was electrospun into a rotating thrombin bath to generate an aligned sheet of electromechanically stretched, Barreto-Ortiz et al., 2013; Zhang et al., 2014; Barreto-Ortiz et al., 2015, heparinized fibrin microfibers. The sheets were wrapped around a mandrel, as described previously, Elliott et al., 2019, ultimately yielding a hollow, heparinized fibrin sdVG. The concentration of LMWH can be varied by altering the ratio of LMWH-F:fibrinogen, keeping the final cumulative concentration at 2.0 wt %.

Following the electrospinning procedure as previously described, Zhang et al., 2009, two-dimensional (2D) sheets for heparin concentration testing were fabricated. Square frames with a side length of 1 cm were flipped through the electrospun sheet to create 15-30 layers (FIG. 3A). The square frames were crosslinked in EDC/NHS; dehydrated using a series of EtOH concentrations; rehydrated by incubation in 75% EtOH for 15 mins and 3 washes in DI H₂O for 5 mins each; and stored at 4° C. The location and concentration of the embedded heparin was further controlled by changing the type and number, respectively, of hydrogel sheets used to fabricate the scaffold (FIG. 3B).

### 1.4 Platelet Adhesion Functional Assay

For all platelet assays, the 3.8% sodium citrate, human or porcine platelet rich plasma (PRP) (BioIVT) was thawed from −80° C. to 4° C. prior to use. The PRP was then incubated with the hydrogel scaffold for 1 hour at 37° C. Stacy et al., 2014.

For the 2D sheets, 500-μL porcine PRP was incubated with 0.1- or 0.5-U/mL thrombin (BioPharm Labs, 91-030) with the sheet in a 24-well, non-tissue culture treated, PDMS coated plate, which was slowly rotated. Samples were rinsed in PBS and fixed for confocal microscopy to assess platelet adhesion. PRP supernatant was collected for the Technothrombin® thrombin generation assay (TGA, Technoclone) to assess platelet activation following the manufacturer's instructions for the RC High reagent. Yao et al., 2020. Bovine collagen I (0.1 mg/mL, Advanced Biomatrix) coated coverslips in a 24-well, tissue culture treated plate served as a positive control for the TGA.

For the 0.6-mm and 5-mm inner diameter grafts, the human PRP was injected into the lumen of the graft in a LumenGen bioreactor (Bangalore Integrated System Solutions Ltd., Bangalore, India) and the bioreactor was slowly rotated inside the incubator to coat all surfaces of the lumen. The hydrogel scaffolds were washed with PBS 3 times to remove unattached platelets.

### 1.5 Immunofluorescence Staining and Confocal Microscopy

The platelets were fixed with 3.7% PFA (Thermo Fisher Scientific, F79-1) for 30 minutes, permeabilized with 0.1% Triton X-100 (Thermo Fisher Scientific, 85111) for 20 minutes, washed with PBS for 3 minutes, and blocked in 1% BSA (Sigma-Aldrich, A3059-50 g) overnight. Elliott et al., 2019. The samples were washed in PBS, incubated in rabbit anti-CD41 primary antibody overnight at 4° C., washed with PBS, incubated in phalloidin and anti-rabbit secondary antibody for 2 hours at room temperature, washed with PBS, incubated in DAPI for 15 minutes, and washed with PBS. The sheets were then stored in Milli-Q H₂O at 4° C. Finally, the sheets or grafts were imaged using confocal microscopy (Carl Zeiss AG, LSM 780).

### 1.6 Confocal Image Analysis

Platelet quantification was conducted using the spot package in Imaris software (Bitplane). Platelets were identified using a threshold of 5-μm diameter spheres on the fluorescence from phalloidin (FIG. 6). Phalloidin is a marker for filamentous actin (F-actin). The number of platelets identified from F-actin was labelled as activated platelets.

1.7 Implantation of sdVGs

Fibrin and heparinized sdVGs measuring 5-mm inner diameter and 2-cm length with a 500-μm thick heat-treated poly(F-caprolactone) (PCL) surgical sheath, Elliott et al., 2019, were implanted as carotid artery interposition grafts in Yorkshire pigs (46±6 kg) (FIG. 9A). The Institutional Animal Care and Use Committee of The University of Chicago reviewed and approved the protocol (72605). Bilateral interposition surgery was performed whenever possible to reduce animal numbers. The pigs underwent placement of an end-to-end anastomosis of the graft to the carotid artery. DAPT of aspirin (325 mg) and Plavix (75 mg) was administered daily post-op. The endpoint for evaluation was 4 weeks following transplantation, with non-invasive color flow Doppler ultrasound performed 2 and 4 weeks post-op to assess patency. MRI also was performed at post-op week 4 just prior to harvest to assess patency.

### 1.8 Statistical Analysis

Statistical analysis was performed with Prism 9.0.0 (GraphPad Software). Unpaired t tests, one-way ANOVA with Tukey's posttest, or two-way ANOVA with Tukey's or Sidak's posttest were used where appropriate. Unless otherwise indicated, graphical data were reported as mean±SD for sample size larger than one. Significance levels were represented by *p<0.05, **p<0.01, ****p<0.0001.

### Example 2

Off-the-Shelf, Heparinized sdVG Supports Patency and Remodeling in a Porcine Model

### 2.1 Overview

Vascular bypass prostheses development research has been ongoing for over 50 years, but thrombogenicity continues to pose a serious challenge to the clinical translation of engineered grafts. Previously we established natural polymer-based small-diameter vascular grafts (sdVG) composed of fibrin hydrogel microfiber tubes (FMT) with an external poly(8-caprolactone) (PCL) sheath, capable of supporting patency in mice. Towards their clinical translation, we report the FMT's shelf stability, scale-up to a size suitable for human application, and successful conjugation of an antithrombotic to the fibrin scaffold to improve patency in a porcine model. The FMT was stable when stored for up to one year at −20° C., 4° C., and 23° C. with minimal changes in hydrogel mechanical properties and swelling ratio, indicating off-the-shelf availability of the FMT. An external PCL sheath provides mechanical strength for implantation of the FMT in a carotid artery interposition porcine model without rupture. However, one in six Fibrin-PCL grafts and the GORE-TEX® expanded polytetrafluoroethylene control graft had complete lumen occlusion due to clot formation at 2 weeks post-implantation. To reduce thrombogenicity, we conjugated low molecular weight heparin to the protein backbone of the fibrin scaffold, enabling local and sustained anticoagulant delivery. We demonstrate that the low molecular weight heparin embedded in the fibrin scaffold remains active in vitro through platelet adhesion and activation reduction. Heparin conjugation also improved performance in vivo by reducing thrombogenicity and reliably extending the timeframe of patency beyond 2 weeks post-implantation. Patent sdVGs underwent neotissue formation, supporting extensive cell infiltration as the fibrin layer degraded. By 4-5 weeks post-implantation, all four of the heparinized Fibrin-PCL grafts had stenosis due to neointimal hyperplasia Fibrin-PCL comparable to the currently clinically used non-biodegradable GORE® PROPATEN® vascular grafts. This hyperplasia has no relation to the heparin coatings. The presence of endothelial cells on the luminal surface of our sdVGs at 4-5 weeks post-implantation is promising, and incorporation of an anti-proliferative drug may prolong patency and enable the formation of a complete tunica intima. This study establishes a heparinized Fibrin-PCL sdVG with off-the-shelf availability and reduced thrombogenicity, providing a pro-regenerative alternative to autologous bypass vessels with limited availability and thrombotic synthetic polymer scaffolds.

### 2.2 Background

Cardiovascular disease accounts for one-third of deaths worldwide and is the leading cause of death in the United States, resulting in a death every 37 seconds. Satterhwaite et al., 2005; Westein et al., 2013; Atheroscloerosis, 2014; Heart Disease Facts, 2020. Atherosclerosis, or plaque buildup within the vessel wall that restricts or occludes blood flow, is a significant underlying cause of cardiovascular disease. Common presentations include coronary artery disease (CAD), cerebrovascular disease, and peripheral artery disease (PAD). Gallino et al., 2014; Ross, 1999. Standard initial treatments for this disease include lifestyle changes and drug therapies. Westein et al., 2013; Atheroschlerosis, 2014. However, of the 10 million people in the United States who have PAD, 26% of these patients have adverse limb outcomes from continued plaque buildup. Kullo and Rooke, 2016; Varu et al., 2010. The “end-stage” of PAD is critical limb ischemia (CLI), which can lead to surgery for limb salvage, amputation, or death. Kullo and Rooke, 2016; Varu et al., 2010; Norgren et al., 2007. Surgical procedures to restore blood flow include endovascular procedures such as angioplasty, stent insertion, or atherectomy. In patients with severe vascular stenosis (narrowing), arterial bypass surgery re-establishes blood flow in the coronary and peripheral arteries. Bypass surgery is the optimal choice for patients requiring a long-term revascularization solution. Elliot et al., 2019; Houstan et al., 2001. Autografts, like the patient's saphenous vein (SV) or internal thoracic artery (ITA), are the clinical gold standard for bypass grafts. Unfortunately, autografts require a secondary surgical site and are unavailable in patients with widespread atherosclerosis or previously harvested vessels. For CLI patients, secondary vein graft failure occurs in 20% of patients by one year, and inpatient hospital treatment for the first year after bypass costs over $29,000. Varu et al., 2010. Thus, there is an urgent clinical need to develop engineered grafts that provide long-term patency.

To this end, we previously developed natural polymer-based small-diameter vascular grafts (sdVGs, <6 mm in diameter) composed of fibrin hydrogel microfiber tubes (FMT) that mimicked the ECM and supported the formation of a confluent, stable endothelium both in vitro and in vivo. Elliott et al., 2019; Barreto-Ortiz et al., 2013; Zhang et al., 2014; Barreto-Ortiz et al., 2015. With an external, ultrathin poly(8-caprolactone) (PCL) surgical sheath, the FMTs were able to support blood flow and maintain patency for at least 24 weeks as interposition grafts in the abdominal aorta of a mouse. Elliott et al., 2019. The host tissue also remodeled the fibrin scaffold to resemble the native abdominal aorta structural and mechanical features. Elliott et al., 2019. Here, we will assess the FMT shelf-life and Fibrin-PCL sdVG functionality in a large animal model.

Off-the-shelf availability of sdVGs is critical to patients needing emergency arterial bypass. Advantages of off-the-shelf, engineered, acellular sdVGs include increased availability, decreased fabrication costs, decreased potential complications relative to cellularized sdVGs, and no secondary surgical sites. Other important factors for hospitals focused on cost reduction are the storage conditions and product expiration date. Robinson, 2008. Medical device choices, including items for cardiovascular surgery, highly affect hospitals' supply-chain efficiency and revenue. Robinson, 2008. To best serve the patient, surgeon, and hospital, it is crucial to understand the effects of long-term storage on our natural polymer-based scaffolds.

Another impediment and common challenge to clinical translation of engineered grafts is failure due to thrombosis, or clot formation, likely caused by the lack of endothelial barrier function. Bilodeau et al., 2005; Sivarapatna et al., 2015. Prevention of clotting with systemic combination antithrombotic drug therapy treatments is not useful in clinical applications due to increased bleeding complications. Hess et al., 2017. Extensive research focuses on coating the luminal surface of sdVGs with heparin, an anticoagulant drug, to address the thrombosis issue. Dimitrievska et al., 2015; Hoshi et al., 2013; Qiiu et al., 2017. However, heparin-coated vascular stents and grafts only minimally improve outcomes for CAD patients relative to non-coated devices. Haude et al., 2003; Lindholt et al., 2011. Further, the widely, clinically used GORE® PROPATEN® heparin-coated ePTFE graft has a 17% reduced primary patency at 48 months relative to the autologous SV. Dorigo et al., 2011. A more practical, local drug delivery approach combined with a pro-regenerative scaffold is needed to minimize thrombosis in vascular grafts.

We propose to chemically conjugate low molecular weight heparin (LMWH) to the protein backbone of our FMT. This approach permits embedding the LMWH throughout the entire graft, which will yield a more reliable, sustained presence of the anticoagulant drug and thereby reduce graft thrombosis. LMWHs are safer and more effective anticoagulant drugs than unfractionated heparin, both of which are glycosaminoglycans (GAGs). Zhang et al., 2010; Ostadal et al., 2008; Tasatargil et al., 2005. In its active state, LMWH binds to antithrombin III (ATIII) to enhance the ability of ATIII to inactivate coagulation enzymes like thrombin (factor IIa) and the platelet surface factor Xa, thereby preventing platelet activation within the coagulation cascade. Hirsh and Levine, 1992. We compared the patency of Fibrin-PCL and heparinized Fibrin-PCL grafts in a porcine carotid artery interposition model. The commonly used porcine model is excellent for assessing graft function and clinical applicability due to the pig's similarity with the human cardiovascular anatomy, physiology, and thrombosis mechanisms. Pashneh-Tala et al., 2015; Stacy et al., 2014; Hoerstrup et al., 2006. The first 4 weeks were critically important given that grafts undergo maximum thrombus formation during this period. Fleser et al., 2004. Ultimately, we established an off-the-shelf, pro-regenerative sdVG with improved acute patency for arterial bypass applications.

### 2.3 Materials and Methods

### 2.3.1 Storage Assessment of FMTs

Multidirectional Fibrin Grafts were fabricated as previously and dehydrated using increasing, serial ethanol (EtOH) dilutions. Elliott et al., 2019. Dehydrated FMTs were stored in a sealed, light-protected container in either a refrigerator (4° C.), freezer (−20° C.), or room temperature (23° C.) for 1, 3, 6, or 12 months. The temperature and humidity were recorded randomly 1-3 times each week. Control FMTs were tested within 5 days of dehydration and were kept at room temperature. Abdominal aortas from female Fox Chase severe combined immunodeficient Beige mice (CB17.Cg-PrkdcscidLystbg-J/Crl) were used as native control tissue. The Institutional Animal Care and Use Committee of Johns Hopkins University reviewed and approved the protocol for the murine study (MO19E454).

Dehydrated FMTs were also stored in humidity-controlled incubators for accelerated aging. Elevated temperatures of 37° C. were used to simulate longer-term storage at −20° C. and 4° C., while 47° C. was used to simulate storage at 23° C. The accelerated aging time was calculated using the ASTM International F1980-16 standards and a conservative aging factor of 2 33,34. It was assumed that 1 month was 30 days in length. After storage, FMTs were rehydrated and immediately underwent circumferential tensile testing using an electromechanical puller, as previously 10. MatLab (MathWorks) code was used to calculate circumferential ultimate tensile stress (UTS), strain to failure (STF), Young's modulus, modulus of toughness, and modulus of resilience, using:

stress=force/(2*length*wall thickness)

strain=displacement/(inner diameter)

Diameter and wall thickness were calculated from area measurements of cross-sectional images of FMT rings using Image J (NIH) and the assumption that the FMT was circular. Toughness was the area under the stress-strain curve. Young's modulus was the slope of the linearly elastic region before the yield point (R2≥0.95), and the modulus of resilience was the area under this linearly elastic region of the curve. The mass-swelling ratio of the FMT was calculated as the ratio of the wet to dry weight. Caliari and Burdick, 2016.

### 2.3.2 Synthesis of Low Molecular Weight Heparin-Fibrinogen

LMWH was pre-activated by stirring 1 mg/mL LMWH in 0.05M 2-morpholinoethanesulfonic acid (MES, pH=6) in MilliQ H₂O with 1.07 mM N-(3-Dimethylaminopropyl)-N′-ethylcarbodiimide hydrochloride (EDC) and 1.17 mM N-Hydroxysuccinimide (NHS) overnight. Yang et al., 2010. Fibrinogen was solubilized in 10×PBS (pH=˜7.4, 0.83 mg/mL) and mixed with the activated-LMWH solution in a 2:1 ratio for 2 days. Based on elemental analysis, we increased the concentrations of EDC and NHS for carbodiimide crosslinking and ensured LMWH was in large molar excess to fibrinogen (0.0741 mM vs. 0.0016 mM, respectively). Due to LMWH (mean molecular weight 4.5 kDa) being a highly negatively charged molecule, Zhang et al., 2010; Ostadal et al., 2008; Ouyang et al., 1992; Barradell and Buckley, 1992, we altered purification to include centrifugal filtration through a 30 kDa filter at 3500 g until approximately 10% of the original volume remained, to which sucrose was added for a final concentration of 100 mM. Subsequently, we dialyzed this solution through 25 kDa molecular weight cut-off tubing against 100 mM sucrose in reverse osmosis (RO) H₂O for 4 days. Dialysis was performed with sucrose in the tubing and bath to protect the protein during desalinization, drying, and storage. Lee and Timasheff, 1981; Mensink et al., 2017. All synthesis steps were performed at 4° C. Lastly, the LMW,H-fibrinogen (LMWH-F) solution was frozen at −80° C., then lyophilized until dry for storage at 4° C. and future electrospinning.

### 2.3.3 Preparation of Heparinized Fibrin Scaffolds

For electrospinning heparinized fibrin, the concentration of LMWH was controlled by altering the LMWH-F:fibrinogen co-dissolved ratio in 0.2 wt % PEO. Fabrication of 0.6 mm inner diameter FMTs was otherwise performed as previously. Elliott et al., 2019. The location of the drug within the FMT can be altered by modulating which of the longitudinally or circumferentially oriented electrospun fibrin sheets wrapped around the mandrel contain LMWH. The concentration of LMWH in the FMT can be controlled by not only altering the ratio of LMWH-F:fibrinogen used in electrospinning but also by changing the number of fibrin sheets that contain LMWH-F. Here, a 2:3 ratio of LMWH-F:fibrinogen was used to make heparinized scaffolds with LMWH-F incorporated in every layer.

Two-dimensional (2D) heparinized fibrin or fibrin scaffolds were fabricated for in vitro thrombogenicity assays by flipping 1 cm square, 3D-printed frames through the electrospun sheet for a total of 25 layers. After collecting the heparinized fibrin or fibrin sheets, the scaffolds were crosslinked in EDC/NHS overnight; dehydrated using increasing, serial EtOH solutions; and immediately rehydrated without air drying to prevent cracking the sheets. Elliott et al., 2019.

For the 5 mm inner diameter grafts, the path length of the rastering needle was increased to create a 4 cm wide sheet. The fibrin or heparinized fibrin sheets were rolled onto a 5 mm diameter polytetrafluoroethylene (PTFE) mandrel for eight longitudinally oriented layers; one 79 cm long circumferentially oriented layer; and eleven longitudinally oriented layers. The 5 mm inner diameter FMTs were crosslinked with EDC/NHS; dehydrated in increasing, serial EtOH solutions for 30 mins each; and stored at 4° C., as previously described. Elliott et al., 2019. PCL sheaths with 500 μm thick walls were prepared as previously by electrospinning a 16% w/v PCL solution in 10% w/v dimethylformamide (DMF) and 90% w/v dichloromethane (DCM) onto a rotating 8 or 9 mm diameter aluminum mandrel (100 rotations/min). Elliott et al., 2019. The electric field (17 kV) was applied to a 27-gauge blunt-tipped needle with a 6-12 cm air gap between the needle and mandrel. The sheaths were fitted to the FMTs by heat treatment, as previously, Elliott et al., 2019, to ensure no diameter mismatch.

### 2.3.4 Biochemistry

The concentration of LMWH in the heparinized FMTs (0.6 mm inner diameter) was determined using the dimethyl methylene blue (DMMB) colorimetric assay for sulfated GAGs described by Dunham et al., 2021. After measuring the wet and dry weight, the heparinized FMTs were digested in 1 mL of papain solution for 18 hours at 65° C. The digested samples (105 μL/well) and DMMB solution (438 μL/well) were plated on a 96-well plate. The sample absorbance (525 nm) was measured immediately in triplicate using a plate reader. A standard linear curve (adjusted R²≥0.95) made from chondroitin sulfate (0-30 μg/mL in papain, 5 μg/mL increments) was used to calculate the concentration of sulfated GAGs. FMTs were used as a negative control for all drug concentration and release assessments.

A modified DMMB assay was used to quantify the cumulative sulfated GAG release over time via hydrolytic and enzymatic degradation. Saito and Tabata, 2012. To assess the LMWH and total protein released by hydrolytic degradation, heparinized FMTs were incubated in 1 mL of PBS at 37° C. while agitating (100 rpm). Lim et al., 2007; Zhu et al., 2021. The supernatant was exchanged entirely at 1, 2, 4, 8, 24, 48, 96, and 168 hours, then weekly until the sample fully degraded. Accelerated in vitro release was accomplished by incubating samples in 1 mL of 0.5 CU/mL plasmin in PBS at 37° C. while agitating (100 rpm). Barreto-Ortiz et al., 2013; Kamberi et al., 2009; Shen and Burgess, 2012; Matsuzaki et al., 2021. The supernatant was exchanged entirely at 0.5, 1, 2, 4, 8, 12, 24, 36, 48, and 72 hours, then every other day until the sample fully degraded. A standard linear curve (adjusted R²≥0.95) made from LMWH in PBS or plasmin solutions (0-30 μg/mL), as appropriate, was used to calculate the concentration of released sulfated GAGs. For both release assays, the total protein released at each time point was quantified using the Pierce™ BCA Protein Assay Kit following the manufacturer's instructions. The sample absorbance (562 nm) was measured in triplicate using a plate reader, and a standard quadratic curve (adjusted R²≥0.99) made from fibrinogen (0-2000 μg/mL) in PBS or plasmin solutions, as appropriate, was used to calculate the concentration of released protein.

### 2.3.5 In Vitro Thrombogenicity Assessments

To determine if the LMWH remained active in the heparinized scaffolds, 2D scaffolds were incubated in 500 μL of Yorkshire porcine (72,000/μL) or human (24,000/μL) platelet-rich plasma (PRP) with high-purity bovine thrombin (0.1 U/mL) for 1 hour at 37° C. on a gently moving rocker. Fibrin 2D scaffolds were used as a control. All scaffolds were placed in a polydimethylsiloxane (PDMS, 1:7 ratio) coated non-tissue culture treated 24-well plate for incubation. Scaffolds were rinsed three times in PBS to remove non-adhered platelets.

The lactate dehydrogenase (LDH) assay assessed platelet adhesion to the 2D scaffold. Matsuzaki et al., 2021; Yao et al., 2020. Platelets adhered to the scaffolds were lysed by incubating the scaffold in 1 mL of 1% Triton X-100 in PBS for 1 hour at 37° C. Subsequently, 100 μL of the lysis supernatant was combined with 100 μL of the freshly prepared reaction mixture in each well of a flat, clear-bottom 96-well plate. After incubation for 20 minutes at room temperature under light-protected conditions, the sample absorbance (490 nm) was read in triplicate using a plate reader, as directed by the kit manufacturer.

For the Technohrombin® thrombin generation assay (TGA), Yao et al., 2020, 40 μL of PRP supernatant was combined with 10 μL of TGA RC High and 50 μL of TGA Substrate in each well of a MaxiSorp, black 96-well plate. The sample fluorescence (360 nm/460 nm) was read in duplicate for 1 hour at 1-minute intervals at 37° C. using a plate reader, as directed by the kit manufacturer. Bovine collagen type I (0.1 mg/mL) coated glass coverslips in tissue culture plastic 24-well plate were used as a positive control. 2.3.6 Mechanical Testing and Porcine Implantation of sdVGs Fibrin-PCL sdVGs (5 mm inner diameter) underwent circumferential tensile testing using an electromechanical puller following the International Organization for Standardization (ISO) 7198:2016(E) Section A.5.2.4.4 (performed by Nanofiber Solutions Inc.). The radial force was applied at a 50 mm/min rate until failure. In addition to circumferential UTS and STF, maximum circumferential tensile strength (CTS) was calculated as maximum force per unit length divided by 2. Suture retention strength (SRS), or the maximum force required to achieve suture pull-out, was measured following ISO 7198:2016(E) Section A.5.7.4.1. A 6-0 polypropylene monofilament suture (Surgipro™ II, Covidien) was placed through one wall at a distance of 2 mm from the graft end and axially pulled at a rate of 13 mm/min. Heat-treated PCL sheaths, a GORE-TEX® expanded PTFE (ePTFE) graft, GORE® PROPATEN®, porcine native carotid arteries, and porcine native jugular veins were tested as controls. For scanning electron microscopy (SEM), critical point dried FMTs were sputter-coated with platinum for 12 seconds and imaged using an electron microscope.

The Institutional Animal Care and Use Committee of The University of Chicago reviewed and approved the protocol for the porcine study (72605). Bilateral implantations were performed where possible to reduce animal numbers. A GORE-TEX® ePTFE graft and two GORE® PROPATEN® grafts were implanted as clinical controls. Briefly, the pigs were anesthetized by continuous gas anesthesia with isoflurane. The Fibrin-PCL and heparinized Fibrin-PCL sdVGs were implanted in the carotid artery of White Yorkshire x Landrace pigs (45.9±5.2 kg). A portion of the carotid artery was exposed, cross-clamped, and truncated. A 2 cm graft length was inserted as an interposition graft using 6-0 monofilament suture for the end-to-end proximal and distal anastomoses. Finally, the muscle, subcutaneous tissue, and skin were closed with absorbable monofilament sutures. The pigs received heparin (100 U/kg IV) just before clamping the carotid artery to implant sdVGs and dual antiplatelet therapy (DAPT) of aspirin (325 mg/day) and Plavix (75 mg/day) until harvest. Hess et al., 2017.

The endpoints for evaluation were 4 weeks following implantation, with non-invasive color Doppler sonography performed 2 weeks postoperatively compared to the GORE-TEX® ePTFE graft, GORE® PROPATEN® grafts, and native carotid artery controls to assess patency. Circumferential tensile testing was performed within 24 hours on harvested sdVG segments, stored in endothelial cell media at 4° C. until testing. Histology and immunohistochemistry (IHC) were used to assess graft integration and remodeling, as previously. Elliott et al., 2019. Briefly, harvested tissue rings were rinsed and flushed with saline before being fixed with formalin; dehydrated in serial EtOH (70%-100%); embedded in paraffin; serially cross-sectioned at 5 μm along the length; and stained. Hematoxylin and eosin (H&E), Masson's trichrome (MT), Verhoeff van Gieson (VVG), and von Kossa staining were performed by the Johns Hopkins University Oncology Tissue Services and Reference Histology Cores. As previously for IHC staining, Shen et al., 2016, paraffin-embedded tissue sections were primary stained with Rabbit anti-mouse/human CD31 (1:1500) or Rabbit anti-mouse αSMA (1:2000); counterstained with ImmPRESS HRP anti-rabbit IgG, ImmPACT DAB Peroxidase substrate (Vector Laboratories); and hematoxylin stained. Images were taken with an upright light microscope and camera.

### 2.3.7 Statistical Analysis

All porcine implantations of sdVGs were performed with at least 3 biological replicates. The sample size is detailed for each experiment throughout the figure legends. Statistical analysis was performed using GraphPad Prism 9.2.0. Unpaired t-tests, One-Way ANOVA with Tukey's posttest, the mixed-effects model with Tukey's or S̆idák's posttest, or Two-Way ANOVA with Tukey's or S̆idák's posttest were used where appropriate, in which significance levels were set at *p<0.05, **p<0.01, ***p<0.001, and ****p<0.0001. All graphical data were reported as mean±standard deviation unless otherwise indicated.

### 2.3.8 Representative Reagents and Resources

| | | |
|---|---|---|
| Reagent or Resource | Source | Identifier |

Antibodies & Stains

| | | |
|---|---|---|
| Rabbit anti-mouse/human | Abcam | 28364 |
| CD31 | | |
| Rabbit anti-mouse alpha | Abcam | 5694 |
| smooth muscle actin | | |
| Hematoxylin 7211 | Thomas | C860G32 |
| | Scientific | |

Critical Commercial Kits

| | | |
|---|---|---|
| ImmPRESS HRP anti- | Vector | MP-7401 |
| rabbit IgG (peroxidase) | Laboratories | |
| polymer detection kit | | |
| ImmPACT DAB | Vector | SK-4105 |
| peroxidase (HRP) | Laboratories | |
| substrate | | |
| Lactate dehydrogenase | Millipore | 11644793001 |
| cytotoxicity kit | Sigma | |
| Technohrombin ® | Diapharma | 5006235 |
| thrombin generation assay | Group Inc. | |
| Pierce ™ BCA protein | ThermoFisher | 23227 |
| assay kit | Scientific | |
| DOW SYLGARD ™ 184 | Ellsworth | 2065622 |
| silicone encapsulant clear | Adhesives | |
| 3.9 kg kit (PDMS) | | |

Experimental Models: Platelets

| | | |
|---|---|---|
| Porcine platelet-rich | BioIVT | PIG03PL38NCZNG |
| plasma (3.8% NaCit, | | |
| unspecified gender, | | |
| pooled) | | |
| Human platelet-rich | BioIVT | HUMANPL38NCUNG |
| plasma (3.8% NaCit, | | |
| unspecified gender, | | |
| pooled) | | |

Experimental Models: Animals

| | | |
|---|---|---|
| Fox Chase severe | Charles | 250 |
| combined | River, | |
| immunodeficient beige | Frederick, | |
| (CB17.Cg-Prkdc^{scid}Lyst^{bg-J}/ | MD | |
| Crl) | | |
| White Yorkshire × | Oak Hill | Domestic Swine |
| Landrace Pigs | Genetics | |

Software

| | | |
|---|---|---|
| Image J | National | https://imagej.nih.gov/ij/ |
| | Institutes of | |
| | Health | |
| MatLab | Mathworks | https://mathworks.com/products/matlab.html |
| GraphPad Prism 9 | GraphPad | https://www.graphpad.com/ |
| | Software | |
| | Inc. | |
| Adobe Illustrator 2021 | Adobe | https://www.adobe.com/products/illustrator.html |
| BioRender | BioRender | https://biorender.com |

| | | |
|---|---|---|
| Reagent or Resource | Source | Identifier |

Chemicals, Peptides, and Recombinant Proteins

| | | |
|---|---|---|
| Bovine fibrinogen | Millipore Sigma | F8630-5G |
| Poly(ethylene oxide) (Mv4*10⁶) (PEO) | Millipore Sigma | 189464- |
| | | 250G |
| Calcium chloride, anhydrous granular | Millipore Sigma | 902179- |
| | | 100G |
| High-purity bovine thrombin | BioPharm Laboratories | 91-030 |
| N-(3-Dimethylaminopropyl)-N′- | Millipore Sigma | E6383 |
| ethylcarbodiimide hydrochloride (EDC) | | |
| N-Hydroxysuccinimide (NHS) | Millipore Sigma | 130672-5G |
| Poly(ε-caprolactone) (Mn 80,000) (PCL) | Millipore Sigma | 440744- |
| | | 250G |
| Poly(ε-caprolactone) (Mn 45,000) (PCL) | Millipore Sigma | 704105- |
| | | 100G |
| Dimethylformamide (DMF) | Millipore Sigma | 227056-1L |
| Dichloromethane (DCM) | Millipore Sigma | 34856-1L |
| Formalin, 10% Neutral Buffered, with | Sigma-Aldrich | F5304-4L |
| 0.03% Eosin | | |
| Triton X-100 | Millipore Sigma | T9284- |
| | | 100 ML |
| Collagen I, bovine | Advanced Biomatrix | 5010-50 mL |
| Antibody diluent | Life Technologies | 003218 |
| Dual endogenous enzyme block | Agilent Technologies | S200389-2 |
| Dulbecco's phosphate-buffered saline | ThermoFisher Scientific | 14200075 |
| (PBS) (10x), no calcium, no magnesium | | |
| Distilled H₂O | ThermoFisher Scientific | 15230-204 |
| 2-morpholinoethanesulfonic acid (MES) | Millipore Sigma | M3651-50G |
| Sodium hydroxide, ACS reagent | Sigma-Aldrich | 415413- |
| (NaOH) | | 100ML |
| Sucrose ACS reagent | Millipore Sigma | S5016-500G |
| Enoxaparin sodium USP and EP | Millipore Sigma | 1235820- |
| reference standards (LMWH) | | 300MG, |
| | | E0180000 |
| Absolute ethanol (200 proof) | Thomas Scientific | C745L09 |
| Plasmin, Human Plasma, Frozen | Athens Research & | 16-16- |
| | Technology | 161213-F |
| 1,9-dimethyl methylene blue (DMMB) | Sigma | 341088-1G |
| Dulbecco's phosphate-buffered saline | ThermoFisher Scientific | 14190144 |
| (PBS) (1x), no calcium, no magnesium | | |
| Chondroitin sulfate | Fisher Scientific | AAJ6615606 |
| Ethylenediaminetetraacetic acid (0.5M | Sigma-Aldrich | E7889- |
| EDTA) | | 100 ML |
| Formic acid (HCOOH) | Sigma-Aldrich | F0507- |
| | | 100 ML |
| L-cysteine Hydrochloride | Sigma-Aldrich | C1276-10G |
| Papain | Sigma-Aldrich | P4762- |
| | | 50MG |
| Sodium formate (CHNaO₂) | Sigma-Aldrich | 247596- |
| | | 500G |
| Sodium phosphate monobasic | Sigma-Aldrich | 567545- |
| (NaH₂PO₄•H₂O) | | 500GM |
| Weigh Paper | Fisher Scientific | 09-898-12A |

| | | |
|---|---|---|
| Reagent or Resource | Source | Identifier |

Equipment

| | | |
|---|---|---|
| Linear stage, 300 mm travel length, 12 mm | Newmark Systems, | ET-300- |
| leadscrew pitch, Nema17 Mdrive motor with | Inc. | 24, 900055, |
| 2048 CPR encoder | | and 250138 |
| Variable speed motor, 0-55 RPM | Amazon | B00JSYAZM2 |
| Lab jack | Chem Glass | CG-3054-14 |
| Syringe pump | Fisher Scientific | 78-0100I |
| ES30P-10 W high voltage power supply | Gamma High | ES30P- |
| | Voltage Research | 10 W/M1225 |
| | Inc. | |
| Variable speed rocker | Santa Cruz | sc-358757 |
| | Biotechnology | |
| Cimarec+ ™ stirring hotplates | ThermoFisher | SP88857108 |
| | Scientific | |
| Forma ™ Steri-Cycle ™ CO₂ incubator, 184L, | ThermoFisher | 370 |
| polished stainless steel | Scientific | |
| −80° C. freezer | ThermoFisher | 993 |
| | Scientific | |
| Mass balance | Mettler Toledo | MS1105DU |
| Fisherbrand ™ analog vortex mixer | Fisher Scientific | 02215365 |
| Sorvall ™ Legend ™ RT+ centrifuge | ThermoFisher | |
| | Scientific | |
| Electromechanical puller | Danish Myo | DMT560 |
| | Technology A/S, | |
| | Aarhus, Denmark | |
| Tensiometer motor drive test stand (Shimpo | Electromatic | FGS100-PXH |
| Instruments) | | |
| 10 lb tensiometer | Nider Shimpo | FGV-10XY |
| SonoSite MicroMaxx portable ultrasound system | SonoSite | |
| SpectraMax M3 plate reader | SpectraMax | |
| Lyophilizer | Labconco | |
| Incubator Shaker Series | New Brunswick | Excella E24 |
| | Scientific | |
| Upright Light Microscope | Nikon | Accuscope 3000, |
| | | Ergonomic E200 |
| | | LED |
| Camera | Nikon | DS-F12 |
| Critical Point Dryer | Tousimis | Samdri 795 |
| Sputter System | Anatech UDA, | Hummer 6.2 |
| | Hayward, CA | |
| Scanning electron microscope (SEM) | TESCAN, JEOL | MIRA3, JSM- |
| | | 6700F |

| | | |
|---|---|---|
| Reagent or Resource | Source | Identifier |

Other Supplies

| | | |
|---|---|---|
| Multipurpose 6061 aluminum rods, 9 mm | McMaster-Carr | 4634T35 |
| diameter, 3 ft long | | |
| PTFE coated mandrels .023″ diameter (aka | Medical | PTFE304- |
| 0.58 mm) × 73″ long | Component | 023-73-3 |
| | Specialists | |
| Syringe filter, PES, 0.22 μm, 30 mm, sterile | CELLTREAT | 229747 |
| 27-gauge blunt tipped needles 0.5″ length | SAI Infusion | B27-50 100 Bulk |
| | Technologies | |
| Norm Ject sterile luer slip syringe; 1 mL | Air-Tite Products | A1 |
| | Co., Inc. | |
| Air-Tite luer lock tip bulk, non-sterile syringes; | Air-Tite Products | MLB5 |
| 5 mL | Co., Inc. | |
| Surgipro ™ II, Covidien 6-0 polypropylene | eSutures.com | VP706MX |
| monofilament suture | | |
| GORE ® PROPATEN ® Vascular Grafts, 5 mm: | eSutures.com | HPT050015A, |
| thin wall | | HPT050010A |
| GORE-TEX ® Stretch (ePTFE) Vascular Graft, | eSutures.com | S0507 |
| 5 mm × 70 cm: standard wall | | |
| Amicon Ultra-15 centrifugal filter unit with | Millipore Sigma | UFC903096 |
| Ultracel-30 membrane | | |
| Spectra/Por 6 dialysis tubing, 25 KDa MWCO | Thomas Scientific | 3787L36 |
| SPECTRA/POR closures orange 75 mm, buoyant | Thomas Scientific | 3787N70 |
| PYREX ® Heavy Duty Beakers 4000 mL | Thomas Scientific | 1003-4L |
| PYREX ® Griffin Low-Form Beakers | Thomas Scientific | 1000-1L |
| NUNC MaxiSorp, black 96-well plate | Amazon | B00K3392M6 |
| Conical tubes, 15 mL | Fisher Scientific | 14-959-70C |
| Conical tubes, 50 mL | Fisher Scientific | 14-959-49A |
| Eppendorf tubes, 1.5 mL | USA Scientific | 1615-5500 |
| Eppendorf tubes, 2.0 mL | USA Scientific | 1620-2700 |
| Eppendorf tubes, 0.5 mL | USA Scientific | 1605-0000 |
| 96 well plate, clear bottom | Corning | 353072 |
| 24 well non-treated plate with lid, individually | CELLTREAT ® | 229524 |
| wrapped, sterile | Scientific Products | |
| 24 well plate | BD Falcon | 3047 |
| Round coverslip: #1.5 thickness, 12 mm | Thomas Scientific | 64-0712 |
| General purpose liquid in glass thermometer | ThermoFisher | 13-201-644 |
| | Scientific | |
| Borosilicate glass disposable serological pipets | Fisher Scientific | 13-678-25E |
| with regular tip, standard length | | |
| Parafilm | Fisher Scientific | 13-374-12 |

### 2.4.1 Shelf-Life of FMTs

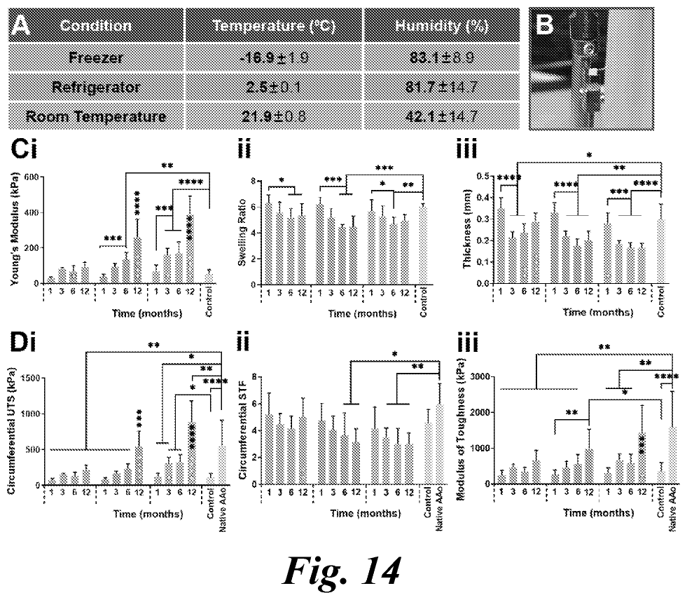

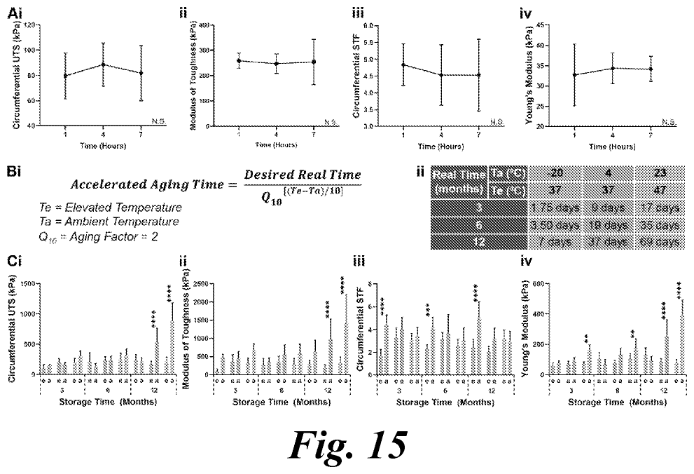

To determine the shelf-life of natural polymer-based sdVGs, we fabricated FMTs as previously, Elliott et al., 2019, and dehydrated them with serial EtOH solutions for long-term storage in the freezer, refrigerator, or room temperature (FIG. 14A). After storage for 1, 3, 6, or 12 months, we rehydrated the FMTs and performed circumferential tensile testing (FIG. 14B). In all cases, the stress-strain curves showed the FMTs were linearly elastic and then often became plastic after the yield point. Mechanical properties of the FMTs were unaffected by the rehydration time (FIG. 15A). This is beneficial for translation as the grafts can be used immediately after rehydration or prepared ahead of time for implantation. Storage in the freezer resulted in the most stable Young's Modulus, or stiffness, over time relative to control FMTs, which were tested within 5 days of dehydration (FIG. 14Ci). Meanwhile, storage in the refrigerator and room temperature significantly increased FMT stiffness by 6 and 3 months, respectively. After 6 months of storage, the mass-swelling ratio, or the relative amount of water the hydrogel scaffold could hold, was significantly reduced for all temperatures (FIG. 14Cii). The combination of increased stiffness and decreased swelling indicate a decreasing mesh size. Caliari and Burdick, 2016. Indeed, the walls were significantly thinner after 3 months of storage (FIG. 14Ciii), which suggests the fibrin microfibers in the biopolymer scaffold may have physically compacted and undergone increased chemical crosslinking leading to the increased stiffness. Caliari and Burdick, 2016; Kanjickal et al., 2008. While the stiffness, swelling ratio, and wall thickness may have changed over 12 months of storage, the maximum reduction in the amount of water the FMTs could hold was only 6.4% (83.4±0.6% and 77.0±3.7% for control FMTs and FMTs in the refrigerator for 12 months, respectively, n=4-13). This high-water retention and the maintained structural integrity indicate the continued functionality of the hydrogel scaffolds after 12 months of storage in all tested temperature conditions.

Next, we further assessed the biological relevance of the mechanical property changes in the stored biopolymer FMTs. We found that storage in the freezer resulted in the most stable circumferential UTS, circumferential STF, and modulus of toughness over time (FIG. 14D). The FMT circumferential UTS was increased by storage in the refrigerator and at room temperature by 12 and 3 months, respectively (FIG. 14Di). However, only the FMTs stored at room temperature for 12 months had increased UTS relative to the native mouse abdominal aorta. The deformability of these FMTs also decreased after 6 and 3 months of storage in the refrigerator and room temperature, respectively, relative to the native mouse abdominal aorta (FIG. 14Dii). For all storage conditions, the modulus of toughness, or the total amount of energy the material absorbed before failure, was reduced relative to the native mouse aorta for at least the first 6 months of storage (FIG. 14Diii). The FMTs stored in the refrigerator or at room temperature for 12 months had an increased modulus of toughness, similar to the native tissue. Therefore, while the changes in FMT mechanical properties may have been statistically significant, these changes were not outside of the range needed to be biologically relevant. Further, the mechanical property changes in the natural polymer-based scaffold were insignificant relative to the mechanical properties of the synthetic polymer surgical sheath previously used for implantations in the mouse abdominal aorta interposition model. Elliott et al., 2019. We found that this PCL sheath has significantly increased circumferential UTS, circumferential STF, and modulus of toughness values of 6,752±1,706 kPa, 9.07±3.03, and 49,285±25,759 kPa, respectively (n=4). This study confirmed the need for the ultrathin, external PCL surgical sheath to provide mechanical properties suitable for implantation.

We used an accelerated aging model to determine if the changes in FMTs mechanical properties resulting from storage could be reproduced in a shorter timescale. In this model, devices are stored at elevated temperatures for short periods to simulate storage at ambient temperatures for more extended periods (FIG. 15B). Levy, 2019; ASTM, 1016. We calculated the accelerated aging time with a conservative aging factor of 2, which is used to simulate a first-order chemical reaction. Caliari and Burdick, 2016. The modeled ambient temperature was within 3.1° C., 1.5° C., and 1.1° C. of the average real freezer, refrigerator, and room temperature conditions. Using this model, we compared the mechanical properties of grafts that underwent accelerated aging to those stored in real-time for 3, 6, and 12 months. We found that the model accurately predicted the circumferential UTS and modulus of toughness at 3 and 6 months (FIG. 15Ci-ii). The model underestimated the circumferential UTS and modulus of toughness at 12 months for the refrigerator and room temperature conditions; the deformability of FMTs stored in the freezer (FIG. 15Ciii); and the stiffness of grafts stored at room temperature, as well as for FMTs stored in the refrigerator for 12 months (FIG. 15Civ). Our findings indicate that the mechanical properties of FMTs can be reliably increased to more closely match native vessel properties using accelerated aging. This study has demonstrated the FMTs have a shelf-life of 12 months. Additionally, the FMTs may be safely stored in an extensive range of temperature conditions with minimal effects on the biopolymer hydrogel scaffold.

### 2.4.2 Development of Heparinized FMTs

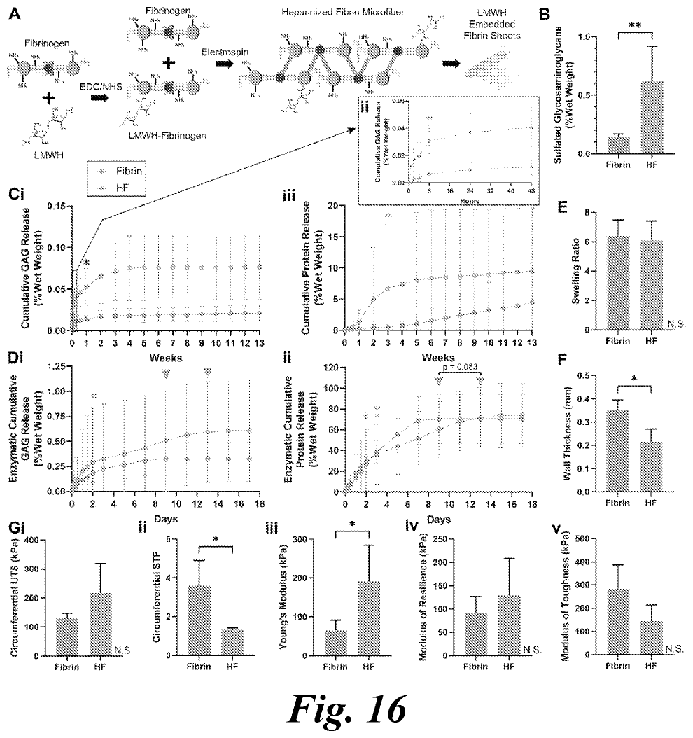

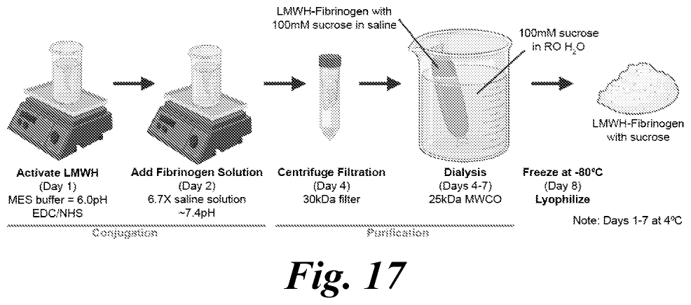

In our previous work, we compared acellular Fibrin-PCL sdVGs to sdVGs seeded with a luminal monolayer of endothelial colony forming cells in an abdominal aorta interposition mouse model and found that endothelialized sdVGs had a more controlled remodeling process with enhanced neotissue formation. Elliott et al., 2019. Other groups have shown that endothelial cells (ECs) are antithrombotic and prevent intimal hyperplasia, Fleser et al., 2004; van Hinsbergh, 2012; Brisbois et al, 2015; Elliott and Gerecht, 2016, critical to sdVG applications. To provide these same benefits while maintaining the off-the-shelf availability of our sdVGs, Ostadal et al., 2008; Tasatargil et al., 2005; Beamish et al., 2009; Saitow et al., 2011, we developed LMWH-embedded sdVGs. We hypothesized that direct conjugation of LMWH to the protein backbone within the fibrin scaffold would allow sustained and local release of the anticoagulant while the scaffold degrades. Fabrication of LMWH-embedded sdVGs first requires synthesis of LMWH-fibrinogen (LMWH-F), which we achieved by conjugation of fibrinogen with LMWH using carbodiimide chemistry (FIG. 16A). Yang et al., 2010; Ouyang et al., 2019. We purified the LMWH-F with centrifuge filtration to remove non-conjugated LMWH and prevent the bulk release of the anticoagulant into the systemic circulation (FIG. 17). We then used dialysis to further purify the LMWH-F by slowly removing saline salts, which enabled electrospinning of the glycoprotein without the arcing caused by charged salt ions. We added sucrose to the dialysis tube and bath to enhance the stability of the protein during desalinization, drying, and storage. Lee and Timasheff, 1981; Mensink et al., 2017. Using this approach, we made the LMWH-F entirely soluble in the electrospinning solution even in the absence of saline, Elliott et al., 2019, thereby enabling us to incorporate LMWH in the fibrin scaffold more efficiently. We next electrospun a 2:3 mixture of LMWH-F:fibrinogen solution into a rotating thrombin bath to generate an aligned sheet of electromechanically stretched, Barreto-Ortiz et al., 2013; Zhang et al, 2014; Barreto-Ortiz et al., 2015, heparinized fibrin microfibers. As previously, Elliott et al., 2019, we wrapped the sheet around a mandrel to yield hollow, heparinized fibrin (HF) microfiber tube. To confirm the presence of LMWH in the HF tubes (0.6 mm inner diameter), we performed a DMMB colorimetric assay for sulfated GAGs. Dunham et al., 2021. We found that the concentration of sulfated GAGs in HF tubes was 4.36 times that in Fibrin tubes (FIG. 16B). These results suggest that the LMWH was successfully bound to the fibrinogen protein and incorporated in significant amounts within the scaffold.

We next assessed the cumulative LMWH and fibrinogen released due to hydrolytic and enzymatic degradation. A modified DMMB assay showed no significant difference in the cumulative release of sulfated GAGs in PBS between the HF and Fibrin tubes until one week (FIG. 16Ci). There was also no significant increase in the cumulative sulfated GAGs released from the HF tube until 8 hours (FIG. 16Cii). The total protein assay showed a significant increase in cumulative proteins released in PBS by HF tubes by 3 weeks relative to the first 4 days (FIG. 16Ciii). There was no significant increase in the cumulative release of sulfated GAGs or protein over time from the Fibrin tubes in PBS, and none of the Fibrin grafts tubes were fully degraded by 13 weeks. While there was no significant difference in protein release in PBS between HF and Fibrin tubes over time due to variability between scaffolds, the HF tubes underwent faster hydrolytic degradation that was physically noticeable by 2 weeks in the form of reduced opacity and structural integrity of the scaffolds. Of the 16 original HF tubes, 10 were still not fully degraded at week 13, and only a portion of the total LMWH was released. Taken together, this indicates there was no burst release of LMWH; instead, an incremental release of LMWH occurred during the hydrolytic degradation of the HF tube.

During enzymatic degradation of the tubes with plasmin in PBS, there was a significant increase in the cumulative sulfated GAGs released by the HF tubes by 2 days, and the release plateaued at 13 days (FIG. 16Di). The LMWH release during enzymatic degradation was incremental over days 2 through 13. The cumulative proteins released by the HF tube in plasmin were significantly increased by day 2 relative to the first 0 to 8 hours; meanwhile, the Fibrin tube did not have a significant increase in cumulative proteins released in plasmin until day 3 (FIG. 16Dii). This indicates that the HF tube initially degraded faster, similar to the hydrolytic degradation case. However, by day 9±1, the Fibrin tube was degraded entirely, and the HF tube did not completely degrade until day 13±5 days (n=6-12). The Fibrin tube tends to degrade faster completely during enzymatic degradation, and LMWH is released until fibrin degradation is complete.

We used the mass-swelling ratio and mechanical properties to assess how the incorporation of LMWH in scaffolds altered graft structural integrity. There was no significant difference in the mass-swelling ratio between the HF and Fibrin tubes (FIG. 16E). While there was no significant difference in inner diameter between HF and Fibrin tubes (data not shown), the HF tubes had significantly thinner walls, with an average difference of 0.137 mm between the groups (FIG. 16F). Despite this difference in wall thickness, there was no significant difference in circumferential UTS between the HF and Fibrin tubes (FIG. 16Gi). The HF tubes were significantly less deformable and, therefore, significantly stiffer than the Fibrin tubes (FIG. 16Gii-iii). There was no significant difference between the two groups regarding the modulus of resilience or toughness (FIG. 16Giv-v). Overall, the HF and Fibrin tubes had very similar mass-swelling ratios and mechanical properties, indicating the incorporation of LMWH did not alter the initial structural integrity.

### 2.4.3 In Vitro Thrombogenicity

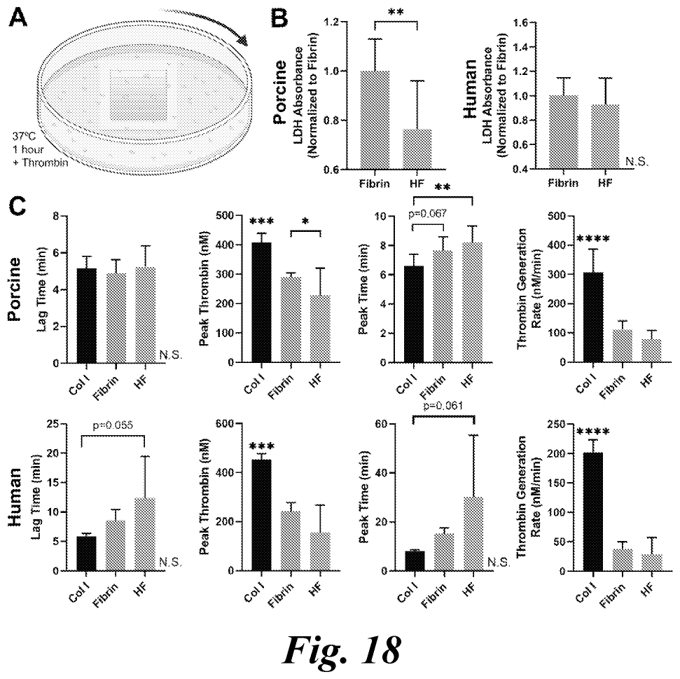

We utilized a dynamic incubation of platelets activated with 0.1 U/mL thrombin on electrospun 2D scaffold sheets as an in vitro thrombogenesis assay (FIG. 18A). Stevens, 2004; Badimon et al., 2012. We measured platelet adhesion to the scaffold using an LDH assay (FIG. 18B). Yao et al., 2020; Shen et al, 2016. We found that the HF scaffolds had significantly reduced LDH absorbance relative to Fibrin scaffolds exposed to porcine PRP, indicating the heparinization substantially reduced porcine platelet adhesion. There was no significant difference in the LDH absorbance between scaffolds exposed to human PRP, indicating a similar number of human platelets adhered to each scaffold. Platelet activation by the scaffold was measured using the sensitive, real-time TGA (FIG. 18C). Yao et al., 2020. For both PRPs, the collagen I coated glass coverslips had substantially increased peak thrombin generation, reduced time to peak thrombin generation, and significantly increased rate of thrombin generation relative to both the Fibrin and HF scaffolds. For porcine PRP, the HF scaffold had significantly reduced peak thrombin generation and slightly delayed time to peak thrombin generation relative to the Fibrin scaffold. For human PRP, the lag time before thrombin generation and time to peak thrombin generation were the most delayed for HF scaffolds. Therefore, the HF scaffolds activated the porcine and human platelets less. The effects were more dramatic for the porcine platelets, which is not surprising given that the porcine PRP contained 3 times as many platelets as the human PRP. These in vitro thrombogenicity assays indicate the potential of our heparinized Fibrin-PCL sdVGs to overcome the thrombogenicity challenge typically faced by synthetic sdVGs. Pashneh-Tala et al., 2015.

2.4.4 Scale-Up of sdVGs

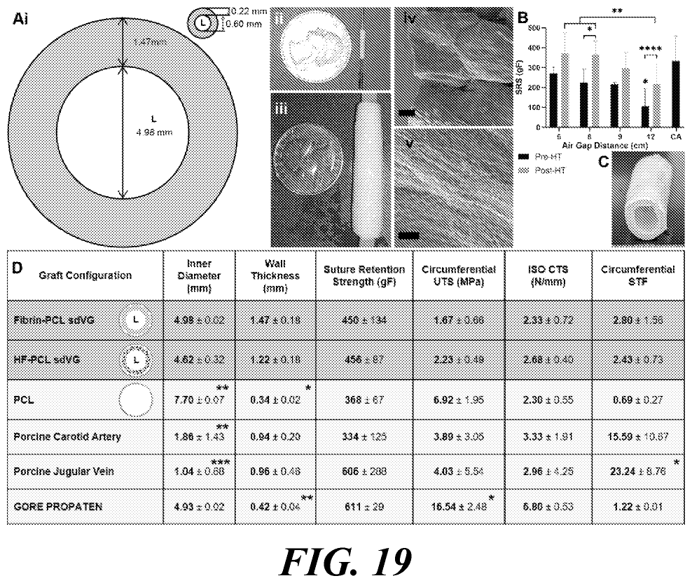

To increase clinical relevancy of the off-the-shelf sdVGs, we scaled-up fabrication of FMTs from 0.6 mm inner diameter and 1 cm length to 5 mm inner diameter and 4 cm length (FIG. 19Ai). By altering the mandrel diameter used to collect the fibrin microfiber sheets, we can match the FMT inner diameter to the patient vessel caliber (FIG. 19Aii-iii). The controlled microfiber topography in our FMTs was previously shown to influence vascular cell organization. Elliott et al., 2019; Barreto-Ortiz et al, 2013; Zhang et al., 2014; Barreto-Ortiz et al., 2013. Using SEM, we confirmed that we could control microfiber alignment in the FMTs with an increased inner diameter (FIG. 19Aiv-v).

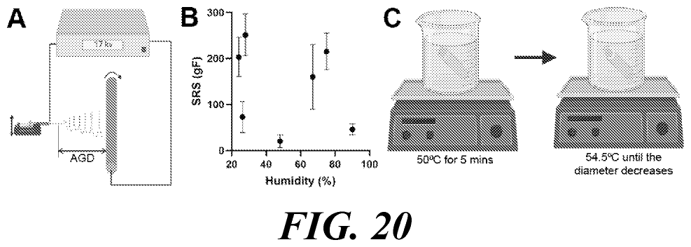

We next optimized the ultra-thin, external PCL surgical sheath. The PCL solution was electrospun at different relative humidities and air gap distances (AGDs) (FIG. 20A). To test SRS at different humidities, all PCL sheaths were electrospun on a 9 mm diameter mandrel with an AGD of 12 cm. According to Nezarati et al., hydrophobic solutions are less likely to be affected by humidity because water vapor does not absorb into hydrophobic jets. Nezarati et al., 2013. Our PCL solution was composed of PCL, DMF, and DCM, with the hydrophobic, organic DCM solvent being the largest portion by volume. It was found that SRS was not affected by humidity (FIG. 20B). Therefore, our results are congruent with Nezarati et al. Important for AGD, fiber diameter is inversely related to the separation distance. Nezarati et al., 2013; Yuan et al., 2004. We tested the SRS for PCL sheaths electrospun with an AGD of 6, 8, 9, and 12 cm (FIG. 19B). Additionally, the sheaths were heat-treated to reduce the inner diameter by 1 mm (FIG. 20C), and SRS was remeasured. Heat treatment of the PCL sheath is essential to shrink the PCL layer onto the fibrin layer, thereby avoiding size mismatch with FMTs. Diameter mismatch causes surgical anastomosis to be more challenging due to the crimping of the sheath, and poor anastomoses can lead to leaks or turbulence in blood flow. Tiwari et al., 2003. We found that a 12 cm AGD significantly decreased SRS for pre- and post-heat treatment sheaths. Additionally, heat treatment significantly increased SRS for sheaths spun with an 8 and 12 cm AGD. The average SRS of post-heat treatment PCL sheaths fabricated at 6 and 8 cm AGD were most similar to native porcine carotid arteries. However, the PCL sheaths fabricated with a 6 cm AGD had only minute changes in diameter in response to heat treatment, resulting in a large diameter mismatch between the PCL sheath and FMT. We determined that the PCL sheath electrospun with an 8 cm AGD onto an 8 mm mandrel was optimal for surgical use due to the high SRS and tight diameter matching the fibrin layer post-heat treatment (FIG. 19C).

We matched the inner diameter of the Fibrin-PCL sdVGs to the pressurized diameter of the native porcine carotid artery, which contracted significantly during surgery and after harvest to 1.86±1.43 mm (FIG. 19D). The sdVGs' wall thickness was similar to the native vessels and significantly thicker than the GORE® PROPATEN® grafts. The PCL sheath was significantly thinner than the native vessels and sdVGs. The fibrin and PCL layers combined yielded a graft with similar SRS, circumferential UTS, ISO CTS, and circumferential STF to the native carotid artery. The GORE® PROPATEN® grafts had significantly increased circumferential UTS relative to all other groups, causing the testing bars to bend while pulling. The jugular vein was significantly more deformable than all the grafts and the PCL sheath. There was no significant difference between the HF-PCL and Fibrin-PCL sdVGs regarding dimensions or mechanical properties. An initial attempt to implant only FMTs without the full-length PCL sheath resulted in rupture and hemorrhage within hours of closure (n=1). Therefore, we used the external, thin PCL sheath, as previously, Elliott et al., 2019, to overcome the mechanical property disadvantages of natural polymer sdVGs. Chan et al., 2018.

2.4.5 Implantation and Patency of sdVGs in Porcine Model

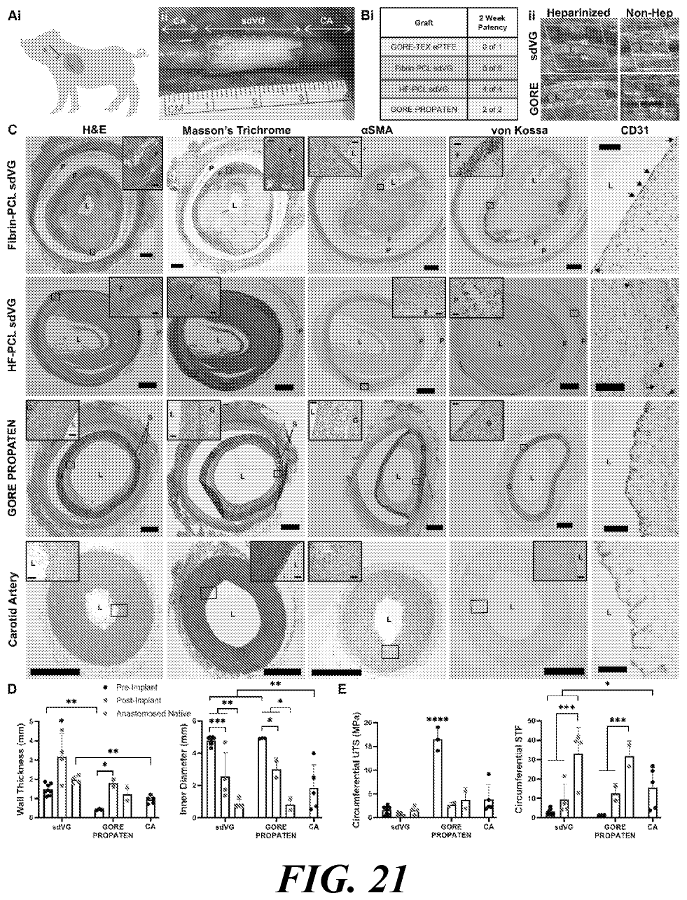

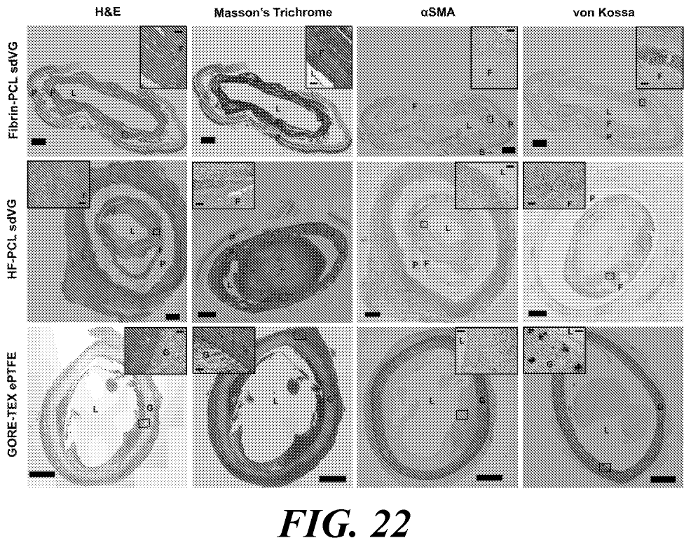

A porcine model enables a more strict thrombogenicity assessment than a murine model, which has different clotting mechanisms than humans. Pashneh-Tala et al., 2015. We implanted a 2 cm length of the sdVGs with a size suitable for human application in the porcine carotid artery as an interposition graft (FIG. 21Ai). We demonstrated the surgical utility of the Fibrin-PCL and HF-PCL sdVGs through end-to-end anastomosis with the porcine carotid artery by vascular surgeons (FIG. 21Aii). All sdVGs supported the high arterial blood pressure without rupture (n=4-6). The pigs received antithrombotic medications like those administered in the clinic, including heparin (100 U/kg, IV) during surgery and post-operative, daily DAPT. Ostadal et al., 2008; What Are Anticoagulants and Antiplatelet Agents, 2017. Most of the Fibrin-PCL and all the HF-PCL sdVGs maintained patency longer than the clinically used GORE-TEX® ePTFE vascular graft (FIG. 21Bi), which was found to be occluded entirely within 2 weeks post-implantation by color Doppler echography (FIG. 21Bii). The GORE-TEX® ePTFE vascular graft and one Fibrin-PCL sdVG were thrombosed (FIG. 22). Due to COVID-19 facility restrictions, we could not assess the patency or harvest two of the Fibrin-PCL sdVGs at 4-5 weeks post-implantation, which is the timeframe in which grafts undergo maximum thrombus formation. Fleser et al., 2004. However, by 9 weeks post-implantation, these two Fibrin-PCL sdVGs had stenosis due to neointimal hyperplasia. All HF-PCL sdVGs were patent at 2 weeks post-implantation but had stenosis due to neointimal hyperplasia by 4 weeks post-implantation, comparable to the clinically used GORE® PROPATEN® grafts. This hyperplasia led to total occlusion in one of the four HF-PCL sdVGs but has no relation to the heparin coatings in the HF-PCL and GORE® PROPATEN® grafts, as shown by the presence of hyperplasia in the Fibrin-PCL sdVGs at 4 and 9 weeks post-implantation. HF-PCL sdVGs had an extended patency time relative to the GORE-TEX® ePTFE and Fibrin-PCL sdVGs, similar to the GORE® PROPATEN® grafts, indicating a reduction of thrombogenicity in vivo.

We assessed patent sdVGs harvested 4-5 weeks after implantation for neotissue formation (FIG. 21C). The PCL sheath was intact and did not seem to have degraded at this early time point. Meanwhile, the fibrin was already being remodeled and degraded, as previously seen in a mouse model. Elliott et al., 2019. There was extensive host cell infiltration and collagen deposition in the fibrin layer of both the Fibrin- and HF-PCL sdVGs (FIG. 21C H&E and MT). The GORE® PROPATEN® scaffold also supported cell infiltration but will not biodegrade. SMCs formed an irregular medial layer in the Fibrin- and HF-PCL sdVGs (FIG. 21C αSMA). This remodeling tissue was not as organized as the native porcine carotid artery medial layer, composed of circumferential SMCs and lamellar units. Neointimal hyperplasia was evident in the Fibrin-PCL, HF-PCL, and GORE® PROPATEN® grafts. Cells that did not stain positive for αSMA, potentially immune cells, were grouped on the luminal side of the fibrin wall layer and GORE® PROPATEN® scaffold. Unsurprisingly, elastin was not visible in Verhoeff van Gieson staining at this early time point (data not shown). Regions of calcification were located in the PCL or where the fibrin luminal surface met infiltrating cells (FIG. 21C von Kossa). Calcification was also visible in the GORE® PROPATEN® and GORE-TEX® ePTFE scaffolds near the graft edges (FIG. 21C von Kossa, S4 von Kossa). Excitingly, ECs were present on the sdVG luminal surface by 4-5 weeks (FIG. 21C CD31). These critical cells will help prevent thrombosis once the LMWH is no longer present in the scaffold. Fleser et al., 2004; van Hinsbergh, 2012; Brisbois et al., 2015; Elliott and Gerecht, 2016. Incorporating an anti-proliferative drug may reduce hyperplasia and enable these ECs to form a more stable tunica intima, ensuring long-term patency. With the extensive host cell remodeling, the harvested HF- and Fibrin-PCL sdVGs had significantly increased wall thickness and decreased inner diameter relative to pre-implant sdVGs (FIG. 21D). A similar trend occurred for the GORE® PROPATEN® grafts. This narrowing of the lumen, yielding a similar diameter to the non-pressurized carotid artery, confirms stenosis from hyperplasia in the sdVGs and GORE® PROPATEN® grafts. The harvested sdVGs maintained similar circumferential UTS and STF relative to pre-implant sdVGs (FIG. 21E), likely due to the PCL sheath. Interestingly, the circumferential UTS of the GORE® PROPATEN® grafts significantly decreased by 4-5 weeks post-implantation, becoming more similar to the native carotid artery and sdVGs. The mechanical properties of the harvested sdVGs and GORE® PROPATEN® grafts were similar to the native carotid artery. Ultimately, the fibrin layer mediated extensive neotissue formation while the PCL sheath maintained structural integrity.

### 2.5 Summary

This study established the off-the-shelf availability of FMTs and anticoagulant embedded sdVGs with a size and mechanical properties suitable for human applications. We show that the FMT, a natural polymer-based scaffold, has a shelf-life of 12 months when stored in the refrigerator, freezer, or at room temperature. This flexibility will ensure the grafts can be easily shipped to and stored by urban and rural hospitals before use in emergency clinical cases. These grafts with off-the-shelf availability could also be used for limb salvage in combat casualties, either as temporary vascular shunts in austere conditions or for definitive vascular repair after evacuation. Rasmussen et al., 2018. While changes in FMT structure caused by long-term storage should be investigated, we found that the scaffold remains functional with a mechanically stable structure that enables immediate implantation after long-term storage. The fresh and stored FMT's reduced strength relative to the native abdominal aorta confirms the need for the synthetic polymer surgical sheath to provide mechanical strength. Interestingly, the accelerated aging of hydrogel scaffolds in controlled temperature, and humidity environments led to the increased strength of the FMT. In the future, we will investigate how this accelerated aging process may be harnessed to alter mechanical properties and eventually remove the need for the synthetic polymer surgical sheath. This would aid the neotissue formation process as our study and others have shown the potential for PCL calcification. De Valence et al., 2012. Computational modeling has previously been used to improve scaffold design and accurately predict clinical outcomes. Szafron et al., 2018; Drews et al., 2020; Lee et al., 2007. It thus should be considered for the optimization of graft design, including geometry and material composition, to balance maintenance of structural integrity with host cell remodeling.

To provide antithrombotic benefits while maintaining off-the-shelf availability of our engineered bypass graft, we developed a heparinized FMT by chemically conjugating LMWH to the scaffold's protein backbone. This novel method for local anticoagulant drug delivery embeds the LMWH throughout the scaffold and enables a more sustained delivery than physical encapsulation techniques, with over 95% of the drug-releasing in 24 hours. Matsuzaki et al., 2021. The drug release profile for the HF tubes indicates no burst release of LMWH occurs during hydrolytic degradation. We anticipated the LMWH would be available as long as the fibrin scaffold was present; indeed, the enzymatic drug release profile indicates that the LMWH is released during the entire 2 weeks of HF tube degradation. Notably, the conjugated LMWH remained active, as shown by the decreased adhesion of porcine platelets to the HF scaffold surface and the reduced porcine and human platelet activation. In vitro thrombogenicity assays with porcine PRP indicated a reduction in the thrombin generation profile that was more substantial than the anticoagulant effect seen in fucoidan coated grafts, which maintained patency in a rabbit model for 4 weeks. Yao et al., 2020. The HF and Fibrin tubes also had similar hydrogel swelling and mechanical properties.

Scale-up of sdVGs to structures sized for human applications required optimizing the PCL sheath for surgical utility, enabling suturability and improved mechanical properties. Electrospinning air gap distance and heat treatment were critical parameters to improve suture retention strength and Fibrin-PCL layer diameter matching. The pre-implant mechanical properties of the larger diameter HF- and Fibrin-PCL sdVGs were similar to the native porcine carotid artery. The need for the PCL sheath to prevent rupture in the large animal model validated the in vitro shelf-life assessments of FMTs. The LMWH in the HF-PCL sdVGs reliably extended patency in the porcine model beyond 2 weeks. By that time, the clinically available GORE-TEX® ePTFE graft and one of the Fibrin-PCL sdVGs were occluded due to thrombus formation. By 4-5 weeks post-implantation, HF-PCL sdVGs were stenosed due to neointimal hyperplasia, similar to the clinically available GORE® PROPATEN® grafts. Based on the in vitro enzymatic degradation cumulative drug release model, we theorize that LMWH was still available in the fibrin layer at 4-5 weeks in vivo, which is desired until a stable tunica intima is formed. Tissue overgrowth on the luminal surface of the Fibrin-PCL sdVGs indicates the hyperplasia leading to severe stenosis has no relation to the embedded LMWH or PROPATEN® coating. Incorporating an anti-proliferative drug like rapamycin may enhance control of the remodeling process by preventing hyperplasia, Yang et al., 2020, reducing stenosis, and prolonging patency beyond 4-5 weeks until a stable tunica intima is formed.

Fibrin mediated neotissue formation, as previously, by supporting extensive host cell infiltration during scaffold degradation. The GORE® PROPATEN® vascular grafts also helped host cell infiltration, but the scaffold will not degrade over time. Patent sdVGs showed that fibrin supported endothelialization by 4-5 weeks post-implantation. The presence of ECs is auspicious for long-term patency after the LMWH is gone. The irregular medial layer and SMC hyperplasia would also benefit from incorporating an anti-proliferative drug. Future efforts should assess the host immune cells, including macrophages, that are involved in acutely remodeling the fibrin and PCL sheath. Ultimately, the HF tube provided an antithrombotic, pro-regenerative scaffold for neotissue formation, while the synthetic polymer layer provided mechanical stability. The HF-PCL sdVG has exciting potential to remodel towards a healthy native vessel structure and thereby overcome limitations of using autologous vascular tissue harvested from the patient and synthetic polymer grafts. Elliott et al., 2019; Matsuzaki et al., 2021. We have anticipated the human condition and developed anticoagulant embedded, biodegradable sdVGs with off-the-shelf availability to mitigate the effects of prothrombotic environments and progress the clinical and commercial utility of our sdVGs.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
ASTM F1980-16 Standard Guide for Accelerated Aging of Sterile Barrier Systems for Medical Devices, American Society for Testing and Materials (2016).
Atherosclerosis (2014). http://www.heart.org/HEARTORG/Conditions/Cholesterol/WhyCholesterolMatters/Atherosclerosis_UCM_305564_Article.jsp#.
Badimon, L., Padro, T. and Vilahur, G. Extracorporeal assays of thrombosis. Platelets and Megakaryocytes: Volume 3, Additional Protocols and Perspectives, 43-57 (2012).
Barradell, L. B. and Buckley, M. M., Nadroparin Calcium. Drugs 44, 858-888 (1992).
Barreto-Ortiz, S. F. et al. A novel in vitro model for microvasculature reveals regulation of circumferential ECM organization by curvature. PLoS One 8, e81061 (2013).
Barreto-Ortiz, S. F. et al. Fabrication of 3-dimensional multicellular microvascular structures. The FASEB Journal 29, 3302-3314 (2015).
S. Barreto-Ortiz, S. et al., A novel in vitro model for microvasculature reveals regulation of circumferential ECM organization by curvature, PLoS One 8(11) e81061 (2013).
Beamish, J. et al., The effects of heparin releasing hydrogels on vascular smooth muscle cell phenotype, Biomaterials 30(31), 6286-6294 (2009).
Bilodeau, K., Couet, F., Boccafoschi, F. and Mantovani, D., Design of a perfusion bioreactor specific to the regeneration of vascular tissues under mechanical stresses. Artificial organs 29, 906-912 (2005).
Bingley, J. et al., Arterial heparan sulfate proteoglycans inhibit vascular smooth muscle cell proliferation and phenotype change in vitro and neointimal formation in vivo, Journal of Vascular Surgery 28(2), 308-318 (1998).
Brisbois, E. et al., Reduction in thrombosis and bacterial adhesion with 7 day implantation of S-nitroso-N-acetylpenicillamine (SNAP)-doped Elast-eon E2As catheters in sheep, J. Mater. Chem. B 3(8), 1639-1645 (2015).
Buttafoco, L. et al. Physical characterization of vascular grafts cultured in a bioreactor. Biomaterials 27, 2380-2389 (2006).
Caliari, S. R. and Burdick, J. A., A practical guide to hydrogels for cell culture, Nature Methods 13, 405 (2016).
Chan, X. et al., Human Pluripotent Stem Cells to Engineer Blood Vessels, in: U. Martin, R. Zweigerdt, I. Gruh (Eds.), Engineering and Application of Pluripotent Stem Cells, Springer International Publishing, Cham, pp. 147-168 (2018).
de Valence, S., et al., Long term performance of polycaprolactone vascular grafts in a rat abdominal aorta replacement model, Biomaterials 33(1), 38-47 (2012).
Dimitrievska, S., et al., Click-coated, heparinized, decellularized vascular grafts, Acta Biomaterialia 13, 177-187 (2015).
Dorigo, W., et al., A multicenter comparison between autologous saphenous vein and heparin-bonded expanded polytetrafluoroethylene (ePTFE) graft in the treatment of critical limb ischemia in diabetics, J Vasc Surg 54(5), 1332-8 (2011).
Drews, J., et al., Spontaneous reversal of stenosis in tissue-engineered vascular grafts, Sci Transl Med 12(537) (2020).
Dunham, C., et al., Increased volume and collagen crosslinks drive soft tissue contribution to post-traumatic elbow contracture in an animal model, Journal of Orthopaedic Research 39(8), 1800-1810 (2021).
Elliott, M. et al. Regenerative and Durable Small-Diameter Graft as an Arterial Conduit. Proceedings of the National Academy of Sciences 116, 12710-12719 (2019).
Elliott, M., and Gerecht, S., Three-dimensional culture of small-diameter vascular grafts, Journal of Materials Chemistry B 4(20), 3443-3453 (2016).
Fleser, P. S. et al. Nitric oxide-releasing biopolymers inhibit thrombus formation in a sheep model of arteriovenous bridge grafts. Journal of vascular surgery 40, 803-811 (2004).
Gallino, A., et al., Non-coronary atherosclerosis, Eur Heart J 35(17), 1112-9 (2014).
Gokhale, K. Heart Surgery in India for $1,583 Costs $106,385 in U.S., www.bloomberg.com/news/articles/2013-07-28/heart-surgery-in-india-for-1-583-costs-106-385-in-u-s- (2013).
Gui, L., Muto, A., Chan, S. A., Breuer, C. K. and Niklason, L. E. Development of decellularized human umbilical arteries as small-diameter vascular grafts. Tissue Engineering Part A 15, 2665-2676 (2009).
Hahn, M. S., McHale, M. K., Wang, E., Schmedlen, R. H. and West, J. L. Physiologic pulsatile flow bioreactor conditioning of poly (ethylene glycol)-based tissue engineered vascular grafts. Annals of biomedical engineering 35, 190-200 (2007).
Haude, M. et al. Heparin-Coated Stent Placement for the Treatment of Stenoses in Small Coronary Arteries of Symptomatic Patients. Circulation 107, 1265-1270 (2003).
Heart Disease Facts, 2020. https://www.cdc.gov/heartdisease/facts.htm.
Hess, C. N. et al. A Structured Review of Antithrombotic Therapy in Peripheral Artery Disease With a Focus on Revascularization. Circulation 135, 2534-2555 (2017).
Hirsh, J. and Levine, M. N., Low molecular weight heparin, Blood 79(1), 1-17 (1992).
Hoerstrup, S. P. et al. Functional Growth in Tissue-Engineered Living, Vascular Grafts. Follow-Up at 100 Weeks in a Large Animal Model Circulation, 114, 1-159-1-166 (2006).
Hoshi, R. et al., The blood and vascular cell compatibility of heparin-modified ePTFE vascular grafts, Biomaterials 34(1), 30-41 (2013).
Houston, P., et al., Homing markers for atherosclerosis: applications for drug delivery, gene delivery and vascular imaging, FEBS letters 492(1-2), 73-77 (2001).
Jeon, O., Kang, S.-W., Lim, H.-W., Hyung Chung, J. and Kim, B.-S. Long-term and zero-order release of basic fibroblast growth factor from heparin-conjugated poly(1-lactide-co-glycolide) nanospheres and fibrin gel. Biomaterials 27, 1598-1607 (2006).
Kamberi, M. et al., A novel accelerated in vitro release method for biodegradable coating of drug eluting stents: Insight to the drug release mechanisms, European Journal of Pharmaceutical Sciences 37(3), 217-222 (2009).
Kanjickal, D. et al., Effects of sterilization on poly(ethylene glycol) hydrogels, Journal of Biomedical Materials Research Part A 87A(3), 608-617 (2008).
Kullo, I. J. and Rooke, T. W., Peripheral Artery Disease, New England Journal of Medicine 374(9), 861-871 (2016).
Lamberth, C. and Dinges, J., Different Roles of Carboxylic Functions in Pharmaceuticals and Agrochemicals, in Bioactive Carboxylic Compound Classes: Pharmaceuticals and Agrochemicals, First Edition. Edited by Clemens Lamberth and Jürgen Dinges. © 2016 Wiley-VCH Verlag GmbH and Co. KGaA. Published 2016 by Wiley-VCH Verlag GmbH and Co. KGaA.
Lee, S.-W. Lee et al., Importance of flow division on transition to turbulence within an arteriovenous graft, Journal of Biomechanics 40(5), 981-992 (2007).
Lee, J. C. and Timasheff, S. N. The stabilization of proteins by sucrose. Journal of Biological Chemistry 256, 7193-7201 (1981).
Lee, Y.-U. et al. Implantation of inferior vena cava interposition graft in mouse model. JoVE (Journal of Visualized Experiments), e51632-e51632 (2014).
Levy, S., Accelerated Age Testing, DDL Testing (2019).
Lim, H. et al., A Novel Technique for Loading of Paclitaxel-PLGA Nanoparticles onto ePTFE Vascular Grafts, Biotechnology Progress 23(3), 693-697 (2007).
Lindholt, J, et al., The Scandinavian Propaten(®) trial—1-year patency of PTFE vascular prostheses with heparin-bonded luminal surfaces compared to ordinary pure PTFE vascular prostheses—a randomised clinical controlled multi-centre trial, European journal of vascular and endovascular surgery: the official journal of the European Society for Vascular Surgery 41(5), 668-73 (2011).
Matsuzaki, S. Miyamoto, H. Miyachi, R. Iwaki, T. Shoji, K. Blum, Y.-C. Chang, J. Kelly, J. W. Reinhardt, H. Nakayama, C. K. Breuer, T. Shinoka, Improvement of Novel Small-Diameter Tissue Engineered Arterial Graft with Heparin Conjugation, The Annals of Thoracic Surgery 111(4), 1234-1241 (2021).
Mensink, M. A., Frijlink, H. W., van der Voort Maarschalk, K. and Hinrichs, W. L. J. How sugars protect proteins in the solid state and during drying (review): Mechanisms of stabilization in relation to stress conditions. European Journal of Pharmaceutics and Biopharmaceutics 114, 288-295 (2017).
Mozaffarian D, B. E., Go A S, Arnett D K, Blaha M J, Cushman M, de Ferranti S, Despres J-P, Fullerton H J, Howard V J, Huffman M D, Judd S E, Kissela B M, Lackland D T, Lichtman J H, Lisabeth L D, Liu S, Mackey R H, Matchar D B, McGuire D K, Mohler E R 3rd, Moy C S, Muntner P, Mussolino M E, Nasir K, Neumar R W, Nichol G, Palaniappan L, Pandey D K, Reeves M J, Rodriguez C J, Sorlie P D, Stein J, Towfighi A, Turan T N, Virani S S, Willey J Z, Woo D, Yeh R W, Turner M B B; on behalf of the American Heart Association Statistics Committee and Stroke Statistics Subcommittee. Heart disease and stroke statistics-2015 update: a report from the American Heart Association. Circulation 131, e29-322 (2015).
Neumann, T., Nicholson, B. S. and Sanders, J. E. Tissue engineering of perfused microvessels. Microvascular research 66, 59-67 (2003).
Nezarati, R., Eifert, M. B., Cosgriff-Hernandez, E., Effects of Humidity and Solution Viscosity on Electrospun Fiber Morphology, Tissue Engineering Part C: Methods 19(10), 810-819 (2013).
Niklason, L. et al. Functional arteries grown in vitro. Science 284, 489-493 (1999).
Norgren, L. et al., Inter-society consensus for the management of peripheral arterial disease (TASC II), European Journal of Vascular and Endovascular Surgery 33(1), S1-S75 (2007).
Ostadal, P., Alan, D., Vejvoda, J., Segethova, J. and Kruger, A. Anti-Xa activity of enoxaparin and nadroparin in patients with acute coronary syndrome. Exp Clin Cardiol 13, 175-178 (2008).
Ouyang, Y. et al. Systematic analysis of enoxaparins from different sources with online one- and two-dimensional chromatography. Analyst 144, 3746-3755 (2019).
Pashneh-Tala, S., MacNeil, S. and Claeyssens, F. The Tissue-Engineered Vascular Graft-Past, Present, and Future. Tissue Engineering Part B: Reviews 22, 68-100 (2015).
Qiu, X. et al., End-point immobilization of heparin on plasma-treated surface of electrospun polycarbonate-urethane vascular graft, Acta Biomaterialia 51, 138-147 (2017).
Rasmussen, T., Wartime Vascular Injury, Military Medicine 183(suppl_2), 101-104 (2018).
Robinson, J., Value-Based Purchasing For Medical Devices, Health Affairs 27(6), 1523-1531 (2008).
Ross, R., Atherosclerosis—an inflammatory disease, The New England Journal of Medicine 340(2), 115-26 (1999).
Saito, T. and Tabata, Y., Preparation of gelatin hydrogels incorporating low-molecular-weight heparin for anti-fibrotic therapy, Acta Biomaterialia 8(2), 646-652 (2012).
Saitow, C., Kaplan, D. L., Castellot, J. J., Heparin stimulates elastogenesis: Application to silk-based vascular grafts, Matrix Biology 30(5), 346-355 (2011).
Satterthwaite, G. et al., Differential gene expression in coronary arteries from patients presenting with ischemic heart disease: further evidence for the inflammatory basis of atherosclerosis, American heart journal 150(3), 488-499 (2005).
Shen, J. and Burgess, D. J., Accelerated in-vitro release testing methods for extended-release parenteral dosage forms, Journal of Pharmacy and Pharmacology 64(7), 986-996 (2012).
Shen, Y.-I. et al., Engineered human vascularized constructs accelerate diabetic wound healing, Biomaterials 102, 107-119 (2016).
Sivarapatna, A. et al. Arterial specification of endothelial cells derived from human induced pluripotent stem cells in a biomimetic flow bioreactor. Biomaterials 53, 621-633 (2015).
Smith, P. K., Mallia, A. K. and Hermanson, G. T. Colorimetric method for the assay of heparin content in immobilized heparin preparations. Analytical biochemistry 109, 466-473 (1980).
Stacy, M. R. et al. Targeted imaging of matrix metalloproteinase activity in the evaluation of remodeling tissue-engineered vascular grafts implanted in a growing lamb model. The Journal of Thoracic and Cardiovascular Surgery 148, 2227-2233 (2014).
Stevens, J. M. Platelet adhesion assays performed under static conditions. Platelets and Megakaryocytes: Volume 1: Functional Assays, 145-151 (2004).
Sundaram, S., Echter, A., Sivarapatna, A., Qiu, C. and Niklason, L. Small-diameter vascular graft engineered using human embryonic stem cell-derived mesenchymal cells. Tissue Engineering Part A 20, 740-750 (2014).
Szafron, J. et al., Immuno-driven and Mechano-mediated Neotissue Formation in Tissue Engineered Vascular Grafts, Annals of biomedical engineering 46(11), 1938-1950 (2018).
Tasatargil, A., Ogutman, C., Golbasi, I., Karasu, E. and Dalaklioglu, S. Comparison of the Vasodilatory Effect of Nadroparin, Enoxaparin, Dalteparin, and Unfractioned Heparin in Human Internal Mammary Artery. Journal of Cardiovascular Pharmacology 45, 550-554 (2005).
Thompson, C. A. et al. A novel pulsatile, laminar flow bioreactor for the development of tissue-engineered vascular structures. Tissue engineering 8, 1083-1088 (2002).
Tiwari, A., Cheng, K. S., Salacinski, H., Hamilton, G., Seifalian, A. M., Improving the patency of vascular bypass grafts: The role of suture materials and surgical techniques on reducing anastomotic compliance mismatch, European Journal of Vascular and Endovascular Surgery 25(4), 287-295 (2003).
van Hinsbergh, V. W., Endothelium-role in regulation of coagulation and inflammation, Seminars in immunopathology, Springer, pp. 93-106 (2012).
Varu, V. N., Hogg, M. E., Kibbe, M. R., Critical limb ischemia, Journal of vascular surgery 51(1), 230-241 (2010).
Westein, E., Flierl, U., Hagemeyer, C. E., Peter, K., Destination known: targeted drug delivery in atherosclerosis and thrombosis, Drug Development Research 74(7), 460-471 (2013).
What Are Anticoagulants and Antiplatelet Agents?, 2017. https://www.heart.org/-/media/data-import/downloadables/1/b/2/pe-abh-what-are-anticoagulants-and-antiplatelet-agents-ucm_300338.pdf.
Williams, C. and Wick, T. M. Perfusion bioreactor for small diameter tissue-engineered arteries. Tissue engineering 10, 930-941 (2004).
Yang Q. et al., A novel biodegradable external stent regulates vein graft remodeling via the Hippo-YAP and mTOR signaling pathways, Biomaterials 258, 120254 (2020).
Yang, H. S. et al. Heparin-Conjugated Fibrin as an Injectable System for Sustained Delivery of Bone Morphogenetic Protein-2. Tissue Engineering Part A 16, 1225-1233 (2010).
Yao, L., Swartz, D. D., Gugino, S. F., Russell, J. A. and Andreadis, S. T., Fibrin-Based Tissue-Engineered Blood Vessels: Differential Effects of Biomaterial and Culture Parameters on Mechanical Strength and Vascular Reactivity. Tissue Engineering 11, 991-1003 (2005).
Yao, Y., Zaw, A. M., Anderson, D. E. J., Hinds, M. T. and Yim, E. K. F. Fucoidan functionalization on poly(vinyl alcohol) hydrogels for improved endothelialization and hemocompatibility. Biomaterials 249, 120011 (2020).
Yuan, X., Zhang, Y., Dong, C., Sheng, J., Morphology of ultrafine polysulfone fibers prepared by electrospinning, Polymer International 53(11), 1704-1710 (2004).
Zhang, F., Zhang, Z. and Linhardt, R. J. in Handbook of Glycomics (eds Richard D. Cummings and J. Michael Pierce) 59-80 (Academic Press, 2010).
Zhang, S. et al. Creating polymer hydrogel microfibres with internal alignment via electrical and mechanical stretching. Biomaterials 35, 3243-3251 (2014).
Zhang, X. et al. Dynamic culture conditions to generate silk-based tissue-engineered vascular grafts. Biomaterials 30, 3213-3223 (2009).
Zhu T., et al., Covalent grafting of PEG and heparin improves biological performance of electrospun vascular grafts for carotid artery replacement, Acta Biomaterialia 119, 211-224 (2021).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing a vascular graft, the method comprising:
(a) conjugating one or more therapeutic agents to a protein to form a therapeutic agent-protein conjugate;
(b) electrospinning a mixture of the therapeutic agent-protein conjugate and the protein to form a plurality of microfibers having the one or more therapeutic agents embedded therein;
(c) forming one or more sheets comprising the plurality of microfibers having the one or more therapeutic agents embedded therein; and
(d) forming a hollow tube comprising the one or more sheets of the plurality of microfibers having the one or more therapeutic agents embedded therein.

2. The method of claim 1, wherein the one or more therapeutic agents comprises a compound having at least one carboxyl group.

3. The method of claim 1, wherein the one or more therapeutic agents is selected from the group consisting of an anticoagulant, an antiplatelet, an antihistamine, an antihypertensive, a nonsteroidal anti-inflammatory drug (NSAID), a statin, an antibiotic, a growth factor, factor Xa inhibitors, direct thrombin inhibitors, an anti-proliferative drug, and combinations thereof.

4. The method of claim 3, wherein the anticoagulant comprises heparin.

5. The method of claim 4, wherein the heparin comprises a low molecular weight heparin (LMWH).

6. The method of claim 5, wherein the LMWH is selected from the group consisting of bemiparin, nadroparin, reviparin, enoxaparin, parnaparin, certoparin, dalteparin, tinzaparin, ardeparin, and pharmaceutically acceptable salts and combinations thereof.

7. The method of claim 1, wherein the protein is selected from the group consisting of fibrinogen, collagen, elastin, gelatin, hyaluronic acid, and combinations thereof.

8. The method of claim 1, wherein the mixture of the therapeutic agent-protein conjugate is electrospun into a rotating bath.

9. The method of claim 8, wherein the one or more therapeutic agents comprises a LMWH, the protein comprises fibrinogen, and the rotating bath comprises thrombin, thereby forming a heparinized fibrin microfiber.

10. The method of claim 1, further comprising rastering a spinneret back and forth to form the one or more sheets of the plurality of microfibers having the one or more therapeutic agents embedded therein.

11. The method of claim 1, further comprising rolling the one or more sheets of the plurality of microfibers having the one or more therapeutic agents embedded therein to form the hollow tube.

12. The method of claim 11, wherein the hollow core has an inner diameter having a range from about 0.1 mm to about 6 mm.

13. The method of claim 11, further comprising combining or alternating one or more sheet of the plurality of microfibers having the one or more therapeutic agents embedded therein with one or more sheets comprising the protein alone, or sheets comprising one or more additional therapeutic agents.

14. The method of claim 11 or claim 13, wherein the one or more sheets of the plurality of microfibers have a combined thickness having a range from about 5 nm to about 10,000 μm.

15. The method of claim 1, wherein the one or more therapeutic agents comprises a low molecular weight heparin (LMWH) and the protein comprises fibrinogen, and the method further comprises activating the LMWH and then conjugating the activated LMWH with the fibrinogen to form a LMWH-fibrinogen conjugate.

16. The method of claim 14, wherein the LMWH is activated with 1-ethyl-3-[3-dimethylaminopropyl]carbodiimide hydrochloride (EDC)/N-hydroxysuccinimide (NHS).

17. The method of claim 15, further comprising purifying the LMWH-fibrinogen conjugate by centrifugal filtration and dialysis to remove non-conjugated LMWH.

18. The method of claim 17, wherein the dialysis comprises a first solution comprising saline and a second solution against which the dialysis occurs comprising reverse osmosis (RO) H₂O.

19. The method of claim 17, wherein the dialysis comprises a first solution comprising sucrose, polyethylene oxide (PEO), or a combination of sucrose and PEO in saline and a second solution against which the dialysis occurs comprising sucrose, PEO, or a combination of sucrose and PEO in RO H₂O.

20. The method of claim 17 or claim 18, further comprising freezing and lyophilizing the purified LMWH-fibrinogen conjugate to form a powdered LMWH-fibrinogen conjugate.

21. A vascular graft, microfibers, sheet, or hollow tube prepared by the method of any one of claims 1 to 20.

22. A vascular graft comprising one or more sheets or hollow tubes comprising a plurality of microfibers having one or more therapeutic agents embedded therein.

23. The vascular graft of claim 22, wherein the one or more therapeutic agents comprises a compound having at least one carboxyl group.

24. The vascular graft of claim 22, wherein the one or more therapeutic agents is selected from the group consisting of an anticoagulant, an antiplatelet, an antihistamine, an antihypertensive, a nonsteroidal anti-inflammatory drug (NSAID), a statin, an antibiotic, a growth factor, factor Xa inhibitors, direct thrombin inhibitors, an anti-proliferative drug, and combinations thereof.

25. The vascular graft of claim 24, wherein the anticoagulant comprises heparin.

26. The vascular graft of claim 25, wherein the heparin comprises a low molecular weight heparin (LMWH).

27. The vascular graft of claim 26, wherein the LMWH is selected from the group consisting of bemiparin, nadroparin, reviparin, enoxaparin, parnaparin, certoparin, dalteparin, tinzaparin, ardeparin, and pharmaceutically acceptable salts and combinations thereof.

28. The vascular graft of claim 22, wherein the plurality of microfibers further comprise a protein selected from the group consisting of fibrinogen, collagen, elastin, gelatin, hyaluronic acid, and combinations thereof.

29. The vascular graft of claim 22, wherein the vascular graft comprises a tubular scaffold comprising a hollow core surrounded by one or more sheets comprising a plurality of microfibers having one or more therapeutic agents embedded therein.

30. The vascular graft of claim 22, wherein the hollow core has an inner diameter having a range from about 0.1 mm to about 6 mm.

31. The vascular graft of claim 22, wherein the one or more sheets have a combined thickness having a range from about 5 nm to about 10,000 μm.

32. A method for treating vascular damage, the method comprising administering a vascular graft of any one of claims 22 to 31 to a subject having vascular damage.

33. The method of claim 32, wherein the vascular graft is administered by vascular bypass surgery.

34. The method of claim 33, wherein the vascular damage is to an artery or vein.

35. The method of claim 33, wherein the vascular damage is caused by a disease or trauma.

36. The method of claim 35, wherein the disease is selected from the group consisting of congenital cardiovascular defect (CCD), coronary artery disease (CAD), or peripheral artery disease (PAD).

37. A mesh comprising a plurality of microfibers formed by the method of claim 1.

38. A kit comprising a powdered LMWH-fibrinogen conjugate, or reagents for preparing the powdered LMWH-fibrinogen conjugate and solutions for reconstituting the powdered LMWH-fibrinogen conjugate for use in electrospinning.

39. A kit comprising a vascular graft or scaffold prepared by the method of claim 1, wherein the vascular graft or scaffold is in a dehydrated or hydrated state, and optionally solutions for rehydrating the vascular grafts or scaffolds before use.
